SEQUENCE LISTING (8 sequences)

SEQ ID NO: 1 - DNA, 36 bp, Artificial Sequence
  Description of Artificial Sequence Synthetic primer
ttcgagctgctgcccgccccgcccctgtcccctagc

SEQ ID NO: 2 - DNA, 36 bp, Artificial Sequence
  Description of Artificial Sequence Synthetic primer
gctaggggacaggggcggggcgggcagcagctcgaa

SEQ ID NO: 3 - DNA, 21 bp, Artificial Sequence
  Description of Artificial Sequence Synthetic primer
ctcaacgacagcagctcgccc

SEQ ID NO: 4 - DNA, 21 bp, Artificial Sequence
  Description of Artificial Sequence Synthetic primer
ggagacgtggcacctcttgag

SEQ ID NO: 5 - DNA, 25 bp, Artificial Sequence
  Description of Artificial Sequence Synthetic primer
tcctctttccccggctccccactag

SEQ ID NO: 6 - DNA, 25 bp, Artificial Sequence
  Description of Artificial Sequence Synthetic primer
gcaaagcccctctcactccagagct

SEQ ID NO: 7 - DNA, 25 bp, Artificial Sequence
  Description of Artificial Sequence Synthetic primer
ggtaaagctcccgggatccgccaat

SEQ ID NO: 8 - DNA, 25 bp, Artificial Sequence
  Description of Artificial Sequence Synthetic primer
gtggactgtcactttggtggctggc

(19) United States
(12) Patent Application Publication
(10) Pub. No.: US 2008/0268533 A1
(43) Pub. Date: Oct. 30, 2008

(54) Methods and Compositions Utilizing Myc and Gsk3Beta to Manipulate the Pluripotency of Embryonic Stem Cells

(75) Inventors: Stephen Dalton, Athens, GA (US); Allan Sheppard, Cambridge (NZ); Cameron McLean, Hamilton (NZ)

Correspondence Address: SUTHERLAND ASBILL & BRENNAN LLP, 999 PEACHTREE STREET, N.E., ATLANTA, GA, 30309, US

(73) Assignee: University of Georgia Research Foundation, Inc., Athens, GA (US)

(21) Appl. No.: 11/574,280

(22) PCT Filed: Aug. 25, 2005
(86) PCT No.: PCT/US2005/030488

§ 371 Date: Feb. 26, 2007

Related U.S. Application Data
(60) Provisional application No. 60/604,453, filed on Aug. 25, 2004.

(51) Int. Cl. C12N 5/06

(52) U.S. Cl. 435/354; 435/377; 435/366

(57) ABSTRACT

The present invention provides methods for stabilizing pluripotent cells through the transcriptional activation of c-myc. Alternatively, the cells are stabilized through the transcriptional activation of c-myc, and the stabilization of c-myc protein levels. c-myc protein can be stabilized through the inhibition of GSK3β or through other components of the cellular machinery that impact on c-myc stability. The invention contemplates the stabilized pluripotent cells produced using the methods described herein. Methods for the identification of compounds that modulate the stabilization of pluripotent cells through modulating transcriptional activation of c-myc, stabilization of c-myc protein levels, and/or inhibition of GSK3β activity are also contemplated.

### BACKGROUND OF THE INVENTION

1. Field of the Invention

The present invention generally relates to methods of stabilizing and/or proliferating pluripotent cells, the cells created by these methods and their uses thereof.

2. Background Art

Murine ES cells can be maintained as a pluripotent, self-renewing population by LIF/STAT3 and Wnt-dependent signaling pathways, but the mechanism of action of these signaling pathways is not understood in great detail.

Murine embryonic stem (ES) cells are a pluripotent cell population isolated directly from the inner cell mass (ICM) of late pre-implantation embryos (Martin, (1981) Proc. Natl. Acad. Sci. USA, 78:7634-7638; Evans & Kaufman, (1981) Nature, 292:154-156). In the presence of IL-6 family members such as leukemia inhibitory factor (LIF), murine ES cells can be maintained indefinitely in a self-renewing state that closely resembles the pluripotent cells of the ICM (Smith et al., (1988) Nature, 336:688-690; Williams et al., (1988) Nature, 336:684-687; Nichols et al., (1990) Development, 110:1341-1348; Pease et al., (1990) Dev. Biol., 141:344-352). Throughout extended periods of passaging, ES cells retain their broad differentiation potential in vitro and when introduced back into blastocyst-stage embryos, can contribute to normal development of the entire embryo and to the germ line (Robertson et al., (1986) Nature, 323:445-448; Beddington & Robertson (1989) Development, 105:733-737).

LIF signaling in murine ES cells is initiated through dimerization of the cytokine receptors LIF-R and gp130 following their engagement by cytokine (Hibi et al., (1990) Cell, 63:1149-1157; Gearing et al., (1991) EMBO J., 10:2839-2848). This signals the activation of Jak family non-receptor tyrosine kinases that phosphorylate LIF-R and gp130 on tyrosine residues (Boulton et al., (1994) J. Biol. Chem., 269:11648-11655; Ernst et al., (1996) J. Biol. Chem., 292:154-156; Stahl et al., (1994) Science, 263:92-95; Nakamura, et al., (1998) Biochem. Biophys. Res. Comm., 248:22-27). STAT3 is then recruited to the receptor complex where it is phosphorylated by Jak, resulting in its subsequent dimerization, nuclear translocation and target gene activation (Zhong et al., (1994) Science, 264:95-98; Damell, (1997) Science, 277:1630-1635). While ectopic STAT3 activity promotes stem cell self-renewal and maintenance of pluripotency in the absence of LIF (Matsuda et al., (1999) EMBO J., 18:4261-4269), inactivation of STAT3 function in LIF-maintained ES cells promotes spontaneous differentiation (Niwa et al., (1998) Genes Dev., 12:2048-2060). These lines of evidence establish STAT3 as an essential component of the LIF-dependent self-renewal pathway.

Murine ES cells are typically cultured in the presence of fetal bovine serum (FBS) or a defined cocktail of supplements that in conjunction with LIF, support proliferation and stem cell maintenance. While LIF has a well characterized pro-maintenance function, other factors present in serum have generally been overlooked in terms of performing a specific role in promoting self-renewal. Ying and co-workers ((2003) Cell, 115:281-292) recently reported however, that factors in FBS such as bone morphogenic proteins (BMPs) may operate in parallel and in collaboration with LIF to promote self-renewal. In the presence of LIF, BMPs antagonize neural differentiation (Tropepe et al., (2001) Neuron, 30:65-78; Ying et al., (2003) Cell, 115:281-292; Ying et al., (2003) Nat. Biotechnol., 21:183-186) although they promote differentiation into non-neural fates in its absence (Johansson & Wiles, (1995) Mol. Cell. Biol., 15:141-151; Wiles & Johansson, (1999) Exp. Cell Res., 247:241-248; Ying et al., (2003) Cell, 115:281-292; Ying et al., (2003) Nat. Biotechnol., 21:183-186). Maintenance of a stable stem cell state therefore appears to require multiple inputs that impose lineage-specific differentiation blocks (Chambers et al., (2003) Cell, 113:643-655; Mitsui et al., (2003) Cell, 113:631-642; Ying et al., (2003) Cell, 115:281-292).

Although the role of LIF in murine ES cell maintenance is well established, there is little evidence to suggest that it performs a similar role in human ES cells (Thomson et al., (1998) Science, 282:1145-1147; Reubinoff et al., (2000) Nat. Biotechnol., 18:399-404), indicating that alternate self-renewal mechanisms are involved. Sato and co-workers ((2004) Nature Med., 10:55-63) recently postulated a role for Wnt-dependent signaling in self-renewal of human and murine ES cells that functions independently of LIF and STAT3. Moreover, suppression of GSK3β, an antagonist of Wnt signaling, is sufficient to maintain short-term culture of human and murine ES cells in the absence of LIF and Wnt (Sato et al, (2004) Nature Med., 10:55- 63). These observations suggest a common mechanism of self-renewal that may be further applicable to adult stem cell populations that require Wnt-dependent signaling (Reya et al., (2003) Nature, 423:409-414; Reya, (2003) Recent Prog. Horm. Res., 58:283-295; Pazianos et al., (2003) Biotechniques, 35:1240-1247). However, other researchers have found that Wnt stimulates hES cell proliferation, and that Wnt is not sufficient or essential for maintaining long-term hES cell cultures (Dravid et al., (2005) Stem Cells Express, doi:10.1634/stemcells.2005-0034).

Although LIF and Wnt may have roles in maintenance of ES cell self-renewal, little is known about their mechanism of action. With the exception of STAT3, downstream effectors of these pathways have not been defined in ES cells.

There is a need, therefore, to identify methods and compositions for the manipulation of pluripotency of embryonic stem cells.

### SUMMARY OF THE INVENTION

It is an object of the present invention to overcome, or at least alleviate, one or more of the difficulties or deficiencies associated with the prior art.

In that regard, the invention contemplates a method for stabilizing the pluripotency of a cell, comprising (a) providing a pluripotent cell; and (b) activating transcription of c-myc in the cell, to thereby stabilize the pluripotency of the cell. In one embodiment, c-myc is activated independently of LIF signaling and/or independently of Wnt signaling. The invention contemplates that the above-described method can further comprise a step of culturing the cell in serum containing medium after step (b). In another embodiment, the method can comprise a step of using an activator of PI3-kinase signaling. In a further embodiment, the method can comprise a step of contacting the cells with a cell culture environment.

The present invention further encompasses a method for stabilizing the pluripotency of a cell, comprising (a) providing a pluripotent cell; (b) activating transcription of c-myc in the cell; and (c) stabilizing c-myc protein, to thereby maintain the pluripotency of the cell. In one embodiment, c-myc is activated independently of LIF signaling and independently of Wnt signaling. The invention contemplates that the above-described method can further comprise a step of culturing the cell in serum containing medium. In another embodiment, stabilizing c-myc comprises contacting the cell with an activator of PI3-kinase signaling. In a further embodiment, the method can comprise a step of contacting the cells with a cell culture environment.

In one embodiment, c-myc is stabilized by inhibiting the activity of GSK3β. GSK3β activity can be inhibited by contacting the cell with exogenous inhibitors, such as, but not limited to, BIO. GSK3β activity on c-myc can also be inhibited through inhibiting c-myc T58 phosphorylation. Furthermore, activation of GSK3β requires its dephosphorylation on serine 9 (S9), and PP2A may act as a S9 phosphatase. GSK3β activity can further be inhibited through inhibiting the Pin1/PP2A signaling mechanism. In another embodiment, c-myc is stabilized through an interaction with Max.

The invention further encompasses the cell produced using any of the above-described methods.

In another aspect, the present invention provides methods for identifying compounds capable of modulating stabilization of pluripotent cells. One such method comprises a) providing an pluripotent cell, b) contacting the pluripotent cell with a test compound, and c) determining whether GSK3β activity has been modulated in the cell, said modulation being an indication that the compound modulates stabilization of the pluripotent cell. In one embodiment, GSK3β activity has been inhibited. In another embodiment, GSK3β activity has been stabilized or increased. Another method comprises a) providing an pluripotent cell, b) contacting the pluripotent cell with a test compound, and c) determining whether c-myc transcription has been modulated in the cell, said modulation being an indication that the compound modulates stabilization of the pluripotent cell. In one embodiment, c-myc transcription has been activated. In another embodiment, c-myc transcription has been decreased or stabilized. A further method comprises a) providing an pluripotent cell, b) contacting the pluripotent cell with a test compound, and c) determining whether c-myc protein levels have been modulated in the cell, said modulation being an indication that the compound modulates stabilization of the pluripotent cell. In certain aspects of the foregoing, the compound that activates transcription of c-myc and/or stabilizes c-myc protein levels also inhibits activation of GSK3β.

In a further aspect, the invention provides methods for identifying a compound capable of stabilizing a human embryonic stem cell. In one embodiment, the method comprises a) providing a human embryonic stem cell, b) contacting the human embryonic stem cell with a test compound, and c) determining whether GSK3β activity has been inhibited in the cell, said inhibition being an indication that the compound stabilizes the human embryonic stem cell.

In yet another aspect, the invention provides methods for identifying a compound that inhibits the activity of GSK3β. In one embodiment, the method comprises a) contacting GSK3β with a substrate for GSK3β and a test compound, and b) determining whether phosphorylation of the substrate is decreased in the presence of the test compound, said decrease in phosphorylation being an indication that the compound inhibits the activity of GSK3β. In another embodiment, the method comprises a) providing a pluripotent cell expressing GSK3β and a substrate for GSK3β, contacting the pluripotent cell with a test compound, and determining whether phosphorylation of the substrate is decreased in the presence of the test compound, said decrease in phosphorylation being an indication that the compound inhibits the activity of GSK3β. In a further aspect, contacting the pluripotent cell with the compound stabilizes the pluripotent cell.

In another aspect of the invention, the invention encompasses a process for making a compound that modulates stabilization a pluripotent cell, comprising carrying out any of the methods described herein to identify a compound that modulates stabilization of a pluripotent cell, and manufacturing the compound. In one embodiment, the compound stabilizes the pluripotent cell.

### BRIEF DESCRIPTION OF THE DRAWINGS

FIGS. 1A-I show enforced c-myc expression suppresses murine ES cell differentiation. (A) shows the configuration of CAGI-ER constructs used to express potential STAT3 target genes in murine ES cells. c-myc expression was driven by the CAGI promoter as a fusion with the steroid binding domain of the estrogen receptor (ER) for inducible activity and coupled to a puromycin resistance gene (puro^{R}) by an internal ribosome entry site (IRES). (B) and (C) show colony morphology of mycER transfected ES cell colonies grown in the presence or absence of 4OHT six days after LIF withdrawal (20× magnification). (D) shows mycER ES cells that were grown in varying concentrations of LIF (0-1×10³ units/ml), in the presence (closed circles) or absence (open circles) of 4OHT. After 6 days (3 passages) colonies were assayed for AP activity, and data was plotted. Data points represent the average of assays performed in quadruplicate with error bars showing the standard error of the mean. (E) shows mycER cells that were grown in the absence of LIF with (closed circles) or without (open circles) 4OHT, or in the presence of 7.5 units/ml LIF with (closed square) or without (open square) 4OHT. AP activity was assessed over a 15 day period and data was shown as the average of assays performed in duplicate. (F)-(I) show flow cytometry profiles of SSEA1 reactivity on the surface of mycER cells grown for 6 or 12 days in the presence of LIF (+LIF) without 4OHT (−4OHT) or, without LIF (−LIF) in the presence or absence of 4OHT. Signal intensity is plotted logarithmically on the x-axis (FL1 log).

FIGS. 2A-H show that c-myc mRNA and protein are downregulated in response to LIF/Wnt withdrawal. (A) shows evaluation of c-myc transcript levels in murine ES cells and EBs (days 1-7) in relation to markers of differentiation by semi-quantitative RT-PCR analysis. LIF maintained ES cells or EBs (−LIF) generated from ES cells were harvested and mRNA analyzed by RT-PCR analysis using primers designed against Rex1, Oct4, Fgf5, brachyury, c-myc and β-actin. (B) shows whole cell lysates (40 μg total protein) from LIF maintained ES cells and EBs (−LIF; see A) that were resolved by SDS PAGE electrophoresis, blotted onto a nitrocellulose membrane and probed with antibodies raised against c-myc, phospho-T58/S62 c-myc (c-myc^{pT58}), GSK3β, Oct4, and GSK3β. (C) shows an evaluation of kinase activity in corresponding samples performed by immunoprecipitating GSK3β from whole cell lysates (300 μg total protein), using MBP as a substrate. GSK3β activity in LIF-maintained mouse ES cells and during differentiation was quantified by phosphorimaging analysis, and is depicted as a fold increase over initial levels in ES cells. Specificity of the assay was determined by immunoprecipitating GSK3β from day 6 EB cell lysates and performing kinase reactions in the presence of MBP, GSK3β II inhibitor, ethanol (EtOH) as indicated by (+). The presence of the GSK3β antibody in the immunoprecipitation is as indicated (+). (D) shows c-myc levels, T58 phosphorylation and GSK3β activity in ES cells maintained with Wnt3a conditioned medium (CM). ES cells maintained in Wnt3a CM for 5 passages (15 days) were harvested or grown as EBs in the absence of CM for 1 or 7 days. Cell lysates were probed with antibodies directed against c-myc, c-myc^{T58A}, Oct4, Cdk2 and GSK3β kinase assays performed as described in (B) but on extracts prepared from day 7 EBs. The specificity of kinase assays was confirmed as described in (B). (E)-(G) show ES cells maintained in LIF (E) or Wnt3a CM (F) or, NIH 3T3 fibroblasts (G) treated with cycloheximide (+CHX, 10 μM). At 30 minute intervals, cells were harvested and whole cell lysates prepared. Following gel electrophoresis and electroblotting, filters were probed with antibodies raised against c-myc, Cdk2 and cyclin E where indicated. (H) shows whole cell lysates prepared from mycER LIF-maintained ES cells and day 1, 2, 3, 4 EBs (−LIF) that had been treated with the proteosome inhibitor, MG132 (+, 5 μM), or ethanol alone (−) 3 hours prior to harvesting. After SDS PAGE and electroblotting onto a membrane, cell lysates (40 μg total protein) were then probed with antibodies raised against c-myc and HDAC1/2.

FIG. 3 shows the localization of GSK3β in mouse ES cells 4 days after withdrawal of LIF as determined by subcellular fractionation. Immunoblots of mESC extracts were fractionated into “P” (pellet, nuclear), and “S” (supernatant, cytoplasmic) fractions. Equivalent amounts (on a per cell basis) were loaded along with equivalent amounts of “T”, total cell lysate.

FIGS. 4A-C show that an okodaic acid-sensitive phosphatase is required for nuclear localization of GSK3β. Sensitivity of GSK3β^{S9} phosphorylation to phosphatase inhibitors in (A) mESCs and (B) d2 EBs was examined. (C) shows a PP2A-A subunit immunoprecipitation of mESC lysate (300 μg protein) probed with GSK3β and c-myc antibodies. The IP control was a “mock” immunoprecipitation minus the antibody.

FIGS. 5A-I show myc^{T58A}ER cell lines can maintain murine ES cells in the absence of LIF. (A) shows that mutation of T58 (T58A) stabilizes c-myc in the absence of LIF. mycER or myc^{T58A}ER levels were evaluated in LIF-maintained lines or in EBs generated by growth in the absence of LIF over five days (d1-d5) by immunoblot analysis using anti-myc and anti-HDAC1 antibodies (loading control). (B)-(G) show phase contrast images (20× magnification) showing the morphology of myc^{T58A}ER and vector alone transfected clonal cell lines grown in the presence of LIF (+LIF, 1×10³ units/ml; B and E), the absence of LIF and 4OHT (C and F) or, absence of LIF but presence of 10 nM 4OHT (D and G). Images were taken of cells after 14 days of culturing under the conditions specified. 4-6 independent clonal cell lines from each group were evaluated, each generating similar results to that shown. (H) shows vector alone, mycER or myc^{T58A}ER cell lines maintained in the absence of factor (−LIF/4OHT), in the presence of LIF (+LIF, 1×10³ units per ml) or absence of LIF but with 4OHT for 14 days. AP assays were performed in triplicate and results expressed as the standard error of the mean. (I) shows RT-PCR analysis of Oct4, Rex1 and β-actin transcripts in vector alone or myc^{T58A}ER cells grown for 14 days in the presence or absence of LIF and 4OHT.

FIG. 6 shows that activation of GSK3β is required for LY 294002 to promote DE formation in human ES cells. Addition of the GSK3β inhibitor, BIO, but not MeBio, blocks LY 294002 induced DE formation. DE formation is indicated by Q-PCR analysis of the cells, showing Sox17 mRNA levels as a fold-increase over untreated cells. Assays were performed in triplicate and are shown as +/−SEM.

FIGS. 7A and B show that knockdown of GSK3β expression with two specific RNAi molecules, but not a mutant RNAi, blocks the ability of LY 294002 to promote DE formation in human ES cells. Cells were assayed by GSK3β and Sox17 immunocytochemistry at the times indicated.

FIGS. 8A-F show myc^{T58A}ER maintained mouse ES cells retain pluripotency after extended periods in the absence of LIF. myc^{T58A}ER ES cells were maintained in the presence of 4OHT for fourteen days (−LIF) then differentiated as embryoid bodies in either the absence or presence of 4OHT (−LIF). Cells were harvested for each of 7 days (1-7) and analyzed for differentiation status as determined by (A) Northern blot analysis by probing for Oct4, brachyury, Fgf5, ornithine decarboxylase (Odc) and GAPDH or, (B) by probing cell lysates (40 μg total protein), after being resolved by SDS PAGE and transferred to a membrane, with anti c-myc and anti Oct4 antibodies. (C) shows myc^{T58A}ER ES cells that were maintained in the presence of 4OHT for 30 days, in the absence of LIF/Wnt and then allowed to differentiate as EBs for 8 days. Cell lysates were subject to immunoblot analysis, probing with anti c-myc, Oct4 and β-tubulin antibodies. (D)-(F) show myc^{T58A}ER ES cells grown in the presence of 4OHT for 30 days (D) that were stained for AP activity (F) or grown for an additional 7 days in the absence of 4OHT (E).

FIGS. 9A-B show that c-myc is a downstream target of LIF/STAT3 signaling in mouse ES cells. (A) shows myc^{T58A}ER ES cells grown in the presence (+) or absence (−) of 4OHT and/or LIF for the times indicated. Cells were passaged at day 4 and LIF was re-added to cultures from day 5 through to day 8. Gene activity was determined by RT-PCR analysis using primer sets designed against c-myc, mycER, Oct4, and β-actin. (B) shows c-myc is a target of STAT3 in vivo. Chromatin immunoprecipitation (ChIP) analysis with a FLAG monoclonal antibody was performed on cross-linked chromatin from a cell line expressing FLAG-tagged STAT3 (STAT3_{FLAG}). Immunoprecipitated chromatin was then used to determine if STAT3_{FLAG} bound the endogenous c-myc promoter using a specific primer pair that flanks an E2F/E-box (left panel). The same chromatin was used as a template in a PCR reaction using primer for the CDK1 promoter that is not implicated as a STAT3 target gene. Addition of FLAG antibody in ChIPs is as indicated (+). To control for the amount of total chromatin in the ChIP assay, a 1/10⁴ of the total sample for each time point (‘input’) was used in a parallel PCR reaction using c-myc or CDK1 specific primers.

FIGS. 10A-B show that c-myc is essential for self-renewal of murine ES cells. (A) shows myc^{Δ40-178}ER cells grown in the presence of LIF and in the absence (t=0) or presence of 4OHT (25 nM) for 2-6 days. mRNA levels for Oct4, Nanog, brachyury, Sox1 and β-actin were evaluated by RT-PCR analysis. (B) shows the percentage of alkaline phosphatase positive colonies as determined at corresponding sampling times to (A). Data are the average of assays performed in triplicate and expressed as standard error of the mean.

FIGS. 11A-D show that c-myc promotes hESC self-renewal in the absence of MEF-CM and bFGF. (A) shows clonally selected BG01 cell lines transfected with CAG-myc^{t58A}ER transgene that were analyzed by immunoblot analysis using antibodies against myc and cdk2. “Ct” indicates the untransfected control cell line, while “tf” indicates the transfected cell line. (B)-(D) show myc^{t58A}ER transfected hESCs grown for 15 days in the absence of MEF-CM and bFGF in the absence of 4OHT (B), or the presence of 4OHT (C) and (D).

### DETAILED DESCRIPTION OF THE INVENTION

Applicant has demonstrated that c-myc has a central role in maintaining self-renewal and pluripotency of pluripotent cells. Specifically, c-myc maintains self-renewal and pluripotency of human and mouse ES cells.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed, Berlin: Springer-Verlag; in Current Protocols in Molecular Biology, F. M. Ausubel et al., eds., Current Protocols, a joint venture between Greene Publishing Associates, Inc. and John Wiley & Sons, Inc., (1998 Supplement); in Current Protocols in Cell Biology, J. S. Bonifacino et al., Eds., Current Protocols, John Wiley & Sons, Inc. (1999 Supplement); and in Current Protocols in Neuroscience, J. Crawley et al., Eds., Current Protocols, John Wiley & Sons, Inc. (1999 Supplement). It is to be understood that as used in the specification and in the claims, “a” or “an” can mean one or more, depending upon the context in which it is used. Thus, for example, reference to “a cell” can mean that at least one cell can be utilized.

The present invention may be understood more readily by reference to the following detailed description of the preferred embodiments of the invention and the Examples included herein. However, before the present compositions and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman & Moldave (Eds.) 1980 Meth. Enzymol. 65; Miller (ed.) 1972 Experiments in Molecular Genetics, Cold Spring Harbor Laboratory, Cold Spring Harbor, N.Y.; Old & Primrose, 1981 Principles of Gene Manipulation, University of California Press, Berkeley; Schleif & Wensink, 1982 Practical Methods in Molecular Biology; Glover (Ed.) 1985 DNA Cloning Vol. I and II, IRL Press, Oxford, UK; Hames & Higgins (Eds.) 1985 Nucleic Acid Hybridization, IRL Press, Oxford, UK; and Setlow & Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

The invention contemplates a method for stabilizing the pluripotency of a cell, comprising (a) providing a pluripotent cell; and (b) activating transcription of c-myc in the cell, to thereby stabilize the pluripotency of the cell. In one embodiment, c-myc is activated independently of LIF signaling and/or independently of Wnt signaling. The invention contemplates that the above-described method can further comprise a step of culturing the cell in serum containing medium. In another embodiment, the method can further comprise a step of contacting the cell with an activator of PI3-kinase signaling. In a further embodiment, the method can comprise a step of contacting the cells with a cell culture environment.

The present invention further encompasses a method for stabilizing the pluripotency of a cell, comprising (a) providing a pluripotent cell; (b) activating transcription of c-myc in the cell; and (c) stabilizing c-myc protein, to thereby maintain the pluripotency of the cell. In one embodiment, c-myc is activated independently of LIF signaling and independently of Wnt signaling. The invention contemplates that the above-described method can further comprise a step of culturing the cell in serum containing medium. In another embodiment, stabilizing c-myc comprises contacting the cell with an activator of PI3-kinase signaling. In a further embodiment, the method can comprise a step of contacting the cells with a cell culture environment.

In one embodiment, c-myc is stabilized by inhibiting the activity of GSK3β. GSK3β activity can be inhibited by contacting the cell with exogenous inhibitors, such as, but not limited to, BIO. GSK3β activity can also be inhibited through inhibiting c-myc T58 phosphorylation. Furthermore, activation of GSK3β requires its dephosphorylation on S9, and PP2A may act as a S9 phosphatase. GSK3β activity can further be inhibited through inhibiting the Pin1/PP2A signaling mechanism. In another embodiment, c-myc is stabilized through an interaction with Max. In a further embodiment, c-myc is stabilized through contacting the cells with an activator of the PI3-kinase pathway. Activators of the PI3-kinase pathway specifically include molecules or compounds that activate Akt, which phosphorylates and inhibits GSK3β activation.

The invention further encompasses the cell produced using any of the above-described methods.

In another aspect, the present invention provides methods for identifying compounds capable of modulating stabilization of pluripotent cells. One such method comprises a) providing an pluripotent cell, b) contacting the pluripotent cell with a test compound, and c) determining whether GSK3β activity has been modulated in the cell, said modulation being an indication that the compound modulates stabilization of the pluripotent cell. In one embodiment, GSK3β activity has been inhibited. In another embodiment, GSK3β activity has been stabilized or increased. Another method comprises a) providing an pluripotent cell, b) contacting the pluripotent cell with a test compound, and c) determining whether c-myc transcription has been modulated in the cell, said modulation being an indication that the compound modulates stabilization of the pluripotent cell. In one embodiment, c-myc transcription has been activated. In another embodiment, c-myc transcription has been decreased or stabilized. A further method comprises a) providing an pluripotent cell, b) contacting the pluripotent cell with a test compound, and c) determining whether c-myc protein levels have been modulated in the cell, said modulation being an indication that the compound modulates stabilization of the pluripotent cell. In certain aspects of the foregoing, the compound that activates transcription of c-myc and/or stabilizes c-myc protein levels also inhibits activation of GSK3β.

In a further aspect, the invention provides methods for identifying a compound capable of stabilizing a human embryonic stem cell. In one embodiment, the method comprises a) providing a human embryonic stem cell, b) contacting the human embryonic stem cell with a test compound, and c) determining whether GSK3β activity has been inhibited in the cell, said inhibition being an indication that the compound stabilizes the human embryonic stem cell.

In yet another aspect, the invention provides methods for identifying a compound that inhibits the activity of GSK3β. In one embodiment, the method comprises a) contacting GSK3β with a substrate for GSK3β and a test compound, and b) determining whether phosphorylation of the substrate is decreased in the presence of the test compound, said decrease in phosphorylation being an indication that the compound inhibits the activity of GSK3β. In another embodiment, the method comprises a) providing a pluripotent cell expressing GSK3β and a substrate for GSK3β, contacting the pluripotent cell with a test compound, and determining whether phosphorylation of the substrate is decreased in the presence of the test compound, said decrease in phosphorylation being an indication that the compound inhibits the activity of GSK3β. In a further aspect, contacting the pluripotent cell with the compound stabilizes the pluripotent cell.

In another aspect of the invention, the invention encompasses a process for making a compound that modulates stabilization a pluripotent cell, comprising carrying out any of the methods described herein to identify a compound that modulates stabilization of a pluripotent cell, and manufacturing the compound. In one embodiment, the compound stabilizes the pluripotent cell.

Applicants have determined that c-myc has a central role in maintaining self-renewal and pluripotency, however, other factors can also be important for self-renewal of stem cells. For example, other signaling pathways are important, and together they work in combination to suppress differentiation of stem cells. Non-limiting examples of such pathways include the BMP signaling and the pathways of various serum components.

As used herein, the term “c-myc” is used interchangeably with N-myc, and is meant to cover all members of the myc family.

As used herein, the terms “biologically active component” or “bioactive component” and “bioactive factor” refer to any compound or molecule that induces a pluripotent cell to be stabilized in culture, wherein said stabilization is due at least in part to transcriptional activation of c-myc and/or stabilization of c-myc protein. While the bioactive component may be as described below, the term is not limited thereto. The term “bioactive component” as used herein includes within its scope a natural or synthetic molecule or molecules which exhibit(s) similar biological activity.

In one embodiment, the pluripotent cell is a mouse cell. In another embodiment, the pluripotent cell is a human cell. As used herein, the term “pluripotent human cell” encompasses pluripotent cells obtained from human embryos, fetuses or adult tissues. In one embodiment, the pluripotent human cell is a human pluripotent embryonic stem cell. In certain other embodiments, the human pluripotent embryonic stem cell is obtained from a human embryonic stem cell colony. In another embodiment the pluripotent human cell is a human pluripotent fetal stem cell, such as a primordial germ cell. In another embodiment the pluripotent human cell is a human pluripotent adult stem cell, such as, an adult hematopoeitic stem cell. As used herein, the term “pluripotent” refers to a cell capable of at least developing into one of ectodermal, endodermal and mesodermal cells. As used herein the term “pluripotent” refers to cells that are totipotent and multipotent. As used herein, the term “totipotent cell” refers to a cell capable of developing into all lineages of cells. As used herein, the term “multipotent” refers to a cell that is not terminally differentiated. In one embodiment the multipotent cell is a neural precursor cell and the multipotent cell culture is a neural precursor cell culture. The pluripotent cell can be selected from the group consisting of an embryonic stem (ES) cell; an inner cell mass (ICM)/epiblast cell; a primitive ectoderm cell, such as an early primitive ectoderm cell (EPL); a primordial germ (EG) cell, and an embryonal carcinoma (EC) cell. The human pluripotent cells of the present invention can be derived using any method known to those of skill in the art at the present time or later discovered. For example, the human pluripotent cells can be produced using de-differentiation and nuclear transfer methods. Additionally, the human ICM/epiblast cell or the primitive ectoderm cell used in the present invention can be derived in vivo or in vitro. EPL cells may be generated in adherent culture or as cell aggregates in suspension culture, as described in WO 99/53021, herein incorporated by reference.

In certain embodiments, the embryonic stem cell of the invention has a normal karyotype, while in other embodiments, the embryonic stem cell has an abnormal karyotype. In one embodiment, a majority of the embryonic stem cells have an abnormal karyotype. It is contemplated that greater than 50%, 55%, 60%, 65%, 70%, 75%, 80%, 85%, 90% or greater than 95% of metaphases examined will display an abnormal karyotype. In certain embodiments, the abnormal karyotype is evident after the cells have been cultured for greater than 5, 6, 7, 8, 9, 10, 11, 12, 13, 14, 15, or passages. In one embodiment, the abnormal karyotype comprises a trisomy of at least one autosomal chromosome, wherein the autosomal chromosome is selected from the group consisting of chromosomes 1, 7, 8, 12, 14, and 17. In another embodiment, the abnormal karyotype comprises a trisomy of more than one autosomal chromosome, wherein at least one of the more than one autosomal chromosomes is selected from the group consisting of chromosomes 1, 7, 8, 12, 14, and 17. In one embodiment, the autosomal chromosome is chromosome 12 or 17. In another embodiment, the abnormal karyotype comprises an additional sex chromosome. In one embodiment, the karyotype comprises two X chromosomes and one Y chromosome. It is also contemplated that translocations of chromosomes may occur, and such translocations are encompassed within the term “abnormal karyotype.” Combinations of the foregoing chromosomal abnormalities are also encompassed by the invention.

As used herein when referring to a cell, cell line, cell culture or population of cells, the term “isolated” refers to being substantially separated from the natural source of the cells such that the cell, cell line, cell culture, or population of cells are capable of being cultured in vitro. In addition, the term “isolating” is used to refer to the physical selection of one or more cells out of a group of two or more cells, wherein the cells are selected based on cell morphology and/or the expression of various markers.

In one embodiment of the invention, the cells comprise an isolated nucleic acid molecule whose expression activates transcription of c-myc and/or stabilizes c-myc protein. In accordance with the present invention, a nucleic acid molecule can be transformed into a pluripotent cell population to inhibit or activate particular genes or gene products, thereby modulating differentiation of the cells. In one embodiment of the foregoing, the cell is a pluripotent stem cell that comprises an isolated nucleic acid molecule encoding a constitutively active form of c-myc. In other embodiments, c-myc can be activated through mutation or gene duplication as is seen in certain types of cancer. In still other embodiments, c-myc can be activated in other cell types with a wide range of growth factors; as a non-limiting example, cytokines can stimulate the c-myc gene in certain cell types. This particular response to a growth factor is dependent on the cell type, such that different biological responses are incurred in different cell types.

In one embodiment of the present invention, the pluripotent cell culture is stable in culture. As used herein, the terms “stable” and “stabilize” refer to the differentiation state of a cell or cell line. When a cell or cell line is stable in culture, it will continue to proliferate over multiple passages in culture, and in certain embodiments, will proliferate indefinitely in culture; additionally, each cell in the culture is preferably of the same differentiation state, and when the cells divide, typically yield cells of the same cell type or yield cells of the same differentiation state. It is contemplated that in certain embodiments, a stabilized cell or cell line does not further differentiate or de-differentiate if the cell culture conditions are not altered, and the cells continue to be passaged and are not overgrown. In certain embodiments, the cell that is stabilized is capable of proliferation in the stable state indefinitely, or for at least more than 2 passages, more than 5 passages, more than 10 passages, more than 15 passages, more than 20 passages, more than 25 passages, or more than 30 passages in culture. In one embodiment, the cell is stable for greater than 1 year of continuous passaging.

As used herein with respect to proteins, the terms “stable” and “stabilizing” refer to maintaining or increasing the measurable amount of a protein in a cell, by inhibiting degradation of the protein, and/or by increasing synthesis of the protein. In certain embodiments of the above invention, c-myc protein is stabilized by inhibiting the activity of GSK3β, which phosphorylates and leads to the degradation of c-myc. In one embodiment, GSK3is inhibited through inhibiting c-myc T58 phosphorylation. As used herein, the term “inhibiting c-myc T58 phosphorylation” refers to any means known now or later developed that reduce phosphorylation of the T58 residue of c-myc, either directly or indirectly. In other embodiments, other kinases are involved that phosphorylate different amino acid residues. For example, it is possible to block the de-phosphorylation of S62, which immediately follows phosphorylation of T58. S62 de-phosphorylation involves a Pin1/PP2A dependent mechanism. Furthermore, activation of GSK3β requires its dephosphorylation on S9, and PP2A may act as a S9 phosphatase. GSK3β activity can therefore be inhibited by inhibition of the Pin1/PPA2 signaling pathway. GSK3β activity can also be inhibited through inhibition of Fbw7, the ubiquitin ligase that is recruited to c-myc when T58 is phosphorylated. Fbw7 ubiquitinates myc, and when S62 is de-phosphorylated, myc gets turned over.

In addition, the activity of myc can also be inhibited by affecting the binding partner of myc, Max. In order to bind DNA, myc is required to form a complex with Max. Any methods known now or developed in the future that affect binding between myc and Max can be used to stabilize or destabilize myc protein and its activity.

In a further embodiment, c-myc is stabilized through contacting the cells with an activator of the PI3-kinase pathway. As used herein, the term “activator of the PI3-kinase pathway” refers to any molecule or compound that increases the activity of PI3-kinase or any molecule downstream of PI3-kinase in a cell contacted with the activator. The invention encompasses, e.g., PI3-kinase agonists, agonists of the PI3-kinase signal transduction cascade, compounds that stimulate the synthesis or expression of endogenous PI3-kinase, compounds that stimulate release of endogenous PI3-kinase, and compounds that inhibit inhibitors of PI3-kinase activity. In certain embodiments of the foregoing, the inhibitor is selected from the group consisting of cytokines, growth factors, neurotrophic factors, peptides, cytokine receptors, growth factor receptors, ionotropic and metabotropic receptors, transporters including plasma membrane reuptake transporters as well as vesicular transporters, voltage gated ion channels, and ion pumps. Activators of the PI3-kinase pathway specifically include molecules or compounds that activate Akt, which phosphorylates and inhibits GSK3β activation.

Methods or assays for determining whether a test compound modulates stabilization of a pluripotent cell are well-known to those of skill in the art. The use of high throughput test systems so that large numbers of test molecules can be screened within a short amount of time is also contemplated. Non-limiting examples of such assays includes protein-protein binding assays, biochemical screening assays, immunoassays, cell based assays, etc. These assay formats are well known in the art. Moreover, combinations of screening assays can be used to find molecules that stabilize pluripotent cells. The screening assays of the present invention are amenable to screening of chemical libraries and are suitable for the identification of small molecule drug candidates, antibodies, peptides, peptidomimetics, and the like. Chemical libraries include commercially available combinatorial chemistry compound libraries from companies such as, but not limited to, Sigma-Aldrich (St. Louis, Mo.), Arqule (Woburn, Mass.), Enzymed (Iowa City, Iowa), Maybridge Chemical Co. (Trevillett, Cornwall, UK), MDS Panlabs (Bothell, Wash.), Pharmacopeia (Princeton, N.J.), and Trega (San Diego, Calif.).

As used herein, the term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, RT-PCT, in situ hybridization, Western blotting, and immunostaining.

As used herein, “activating transcription” refers to an enhancement of the transcription of DNA into RNA in response to an event in comparison to the situation where the event has not occurred. An increase in transcription can be determined using methods well known to those of ordinary skill in the art, and include, without limitation, Northern blotting, in situ hybridization and RT-PCR.

As described above, c-myc protein can be stabilized by inhibiting the activity of GSK3β, or other target molecules. In certain embodiments, nucleic acids can be used to inhibit expression of a target polynucleotide, for example, through the use of antisense polynucleotides, sense polynucleotides, RNA interference, or ribozymes.

Antisense polynucleotides are thought to inhibit gene expression of a target polynucleotide by specifically binding the target polynucleotide and interfering with transcription, splicing, transport, translation, and/or stability of the target polynucleotide. Methods are described in the prior art for targeting the antisense polynucleotide to the chromosomal DNA, to a primary RNA transcript, or to a processed mRNA. The target regions can include splice sites, translation initiation codons, translation termination codons, and other sequences within the open reading frame.

The term “antisense,” for the purposes of the invention, refers to a nucleic acid comprising a polynucleotide that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. The term “antisense nucleic acid” includes single stranded RNA as well as double-stranded DNA expression cassettes that can be transcribed to produce an antisense RNA. “Active” antisense nucleic acids are antisense RNA molecules that are capable of selectively hybridizing with a primary transcript or mRNA encoding a polypeptide having at least 80% sequence identity with the targeted polypeptide sequence.

The antisense nucleic acid can be complementary to an entire coding strand, or to only a portion thereof. In one embodiment, an antisense nucleic acid molecule is antisense to a “coding region” of the coding strand of a nucleotide sequence. The term “coding region” refers to the region of the nucleotide sequence comprising codons that are translated into amino acid residues. In another embodiment, the antisense nucleic acid molecule is antisense to a “noncoding region” of the coding strand of a nucleotide sequence. The term “noncoding region” refers to 5′ and 3′ sequences that flank the coding region that are not translated into amino acids (i.e., also referred to as 5′ and 3′ untranslated regions). The antisense nucleic acid molecule can be complementary to the entire coding region of mRNA, or can be antisense to only a portion of the coding or noncoding region of an mRNA. For example, the antisense oligonucleotide can be complementary to the region surrounding the translation start site. An antisense oligonucleotide can be, for example, about 5, 10, 15, 20, 25, 30, 35, 40, 45, or 50 nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., (1987) Nucleic Acids. Res., 15:6625-6641). The antisense nucleic acid molecule can also comprise a 2′-o-methylribonucleotide (Inoue et al., (1987) Nucleic Acids Res., 15:6131-6148) or a chimeric RNA-DNA analogue (Inoue et al., (1987) FEBS Lett., 215:327-330).

The antisense nucleic acid molecules of the invention are typically administered to a cell or generated in situ such that they hybridize with or bind to cellular mRNA and/or genomic DNA to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using a vector. To achieve sufficient intracellular concentrations of the antisense molecules, the antisense nucleic acid molecule can be placed under the control of a strong prokaryotic, viral, or eukaryotic promoter in a vector constructs.

The present invention further provides compositions for RNA interference. In this technique, double-stranded RNA or dsRNA derived from the gene to be analyzed is introduced into the target cell. As used herein, “dsRNA” refers to RNA that is partially or completely double stranded. The dsRNAs can be linear or circular in structure. The hybridizing RNAs may be substantially or completely complementary. By “substantially complementary,” is meant that when the two hybridizing RNAs are optimally aligned using the BLAST program as described above, the hybridizing portions are at least 95% complementary. In one embodiment, the dsRNA will be at least 100 base pairs in length. Typically, the hybridizing RNAs will be of identical length with no over hanging 5′ or 3′ ends and no gaps. However, dsRNAs having 5′ or 3′ overhangs of up to 100 nucleotides may be used in the methods of the invention. The dsRNA may comprise ribonucleotides, ribonucleotide analogs such as 2′-O-methyl ribosyl residues, or combinations thereof. See, e.g., U.S. Pat. Nos. 4,130,641 and 4,024,222. A dsRNA polyriboinosinic acid:polyribocytidylic acid is described in U.S. Pat. No. 4,283,393. Methods for making and using dsRNA are known in the art.

Once in a cell, the dsRNA is processed into relatively small fragments and can subsequently become distributed throughout the cell. The dsRNA fragments interact, in a cell, with the corresponding endogenously produced messenger RNA, resulting in the endogenous transcript being specifically broken down (Zamore et al., (2000) Cell, 101:25-33). This process leads to a loss-of-function mutation having a phenotype that, over the period of a generation, may come to closely resemble the phenotype arising from a complete or partial deletion of the target gene. The invention provides for a composition comprising a dsRNA that is substantially identical to a portion of a target gene of the target cell genome. In certain embodiments of the foregoing, the target gene is selected from the group consisting of (a) the polynucleotide sequence encoding GSK3β, and (b) a polynucleotide that hybridizes under stringent conditions to a polynucleotide as defined in (a).

The invention further provides for a composition comprising a dsRNA consisting of (a) a first stand comprising a sequence substantially identical to 19-49 consecutive nucleotides of the polynucleotide sequence encoding GSK3β; and (b) a second strand comprising a sequence substantially complementary to the first strand. In one embodiment, the dsRNA inhibits expression of a protein encoded by a polynucleotide hybridizing under stringent conditions to the polynucleotide sequence encoding GSK3β. In further embodiments, the dsRNA has a single stranded overhang at either or both ends. The invention provides for a nucleic acid molecule comprising a regulatory sequence operatively linked to a nucleotide sequence that is a template for one or both strands of the claimed dsRNA. In one embodiment, the nucleic acid molecule further comprises a promoter flanking either end of the nucleic acid molecule, wherein the promoters drive expression of each individual DNA strand, thereby generating two complementary RNAs that hybridize and form the dsRNA. In another embodiment, the nucleic acid molecule comprises a nucleotide sequence that is transcribed into both strands of the dsRNA on one transcription unit, wherein the sense strand is transcribed from the 5′ end of the transcription unit and the antisense strand is transcribed from the 3′ end, wherein the two strands are separated by 3 to 500 basepairs, and wherein after transcription, the RNA transcript folds on itself to form a hairpin.

As an alternative, ribozymes can be used to reduce expression of a polypeptide. As used herein, the term “ribozyme” refers to a catalytic RNA-based enzyme with ribonuclease activity that is capable of cleaving a single-stranded nucleic acid, such as an mRNA, to which it has a complementary region. Ribozymes (e.g., hammerhead ribozymes described in Haselhoff & Gerlach, (1988) Nature, 334:585-591) can be used to catalytically cleave mRNA transcripts to thereby inhibit translation. A ribozyme having specificity for a nucleic acid can be designed based upon the nucleotide sequence of the cDNA or on the basis of a heterologous sequence to be isolated according to methods taught in this invention. In certain embodiments, the ribozyme will contain a portion having at least 7, 8, 9, 10, 11, 12, 13, 14, 15, 16, 17, 18, 19, or 20 nucleotides, that have 100% complementarity to a portion of the target RNA. Methods for making ribozymes are known to those skilled in the art. See, e.g., U.S. Pat. Nos. 6,025,167; 5,773,260; and 5,496,698.

Methods for the culture of mouse ES cells are well known to those of ordinary skill in the art. The methods discussed herein are primarily directed towards the culture of primate ES cells, and particularly, are directed towards the culture of human ES cells.

The methods of the present invention contemplate that cells may be cultured with a feeder cell or feeder layer. As used herein, a “feeder cell” is a cell that is co-cultured with a target cell and stabilizes the target cell in its current state of differentiation. A feeder layer comprises more than one feeder cell in culture. In one embodiment of the above method, conditioned medium is obtained from a feeder cell that stabilizes the target cell in its current state of differentiation. Any and all factors produced by a feeder cell that allow a target cell to be stabilized in its current state of differentiation can be isolated and characterized using methods routine to those of skill in the art. These factors may be used in lieu of a feeder layer, or may be used to supplement a feeder layer.

The present invention contemplates that the feeder cell can be a freshly plated feeder cell. As used herein, the term “freshly plated” means that the feeder cell has been allowed to attach to the tissue culture dish for less than 2 days. In certain embodiments, the feeder cell has been plated for less than 18 hours, in other embodiments the feeder cell has been plated for less than 10 hours, in other embodiments the feeder cell has been plated for less than 6 hours, and in further embodiments, the feeder cell has been plated for less than 2 hours. In another embodiment, the feeder cell has been plated for approximately 6 to 18 hours.

As used herein, the term “contacting” (i.e., contacting a cell with a compound) is intended to include incubating a compound and the cell together in vitro (e.g., adding the compound to cells in culture). The term “contacting” is not intended to include the in vivo exposure of cells to a compound that may occur naturally in a subject (i.e., exposure that may occur as a result of a natural physiological process). It is contemplated that the pluripotent cell is contacted with an effective amount of a compound to thereby stabilize the cell. As used herein, the term “effective amount” of a compound refers to that concentration of the compound that is sufficient in the presence of the cell culture medium to effect the stabilization of the pluripotent cell in culture for greater than 2 passages, greater than 5 passages, greater than 10 passages, or greater than 25 passages in culture. This concentration is readily determined by one of ordinary skill in the art.

The step of contacting the cell with a compound can be conducted in any suitable manner. For example, the cells may be treated in adherent culture, or in suspension culture. It is understood that the cells treated with a compound may be further treated with one or more cell culture environments to stabilize the cells, or to differentiate the cells further.

As used herein, the term “cell culture environment” refers to a cell culture condition wherein the pluripotent cells or embryoid bodies derived therefrom are induced to stabilize, to partially or fully differentiate, or are induced to form a human cell culture enriched in differentiated cells. In certain embodiments, the cell lineage induced by the cell culture environment will be homogeneous in nature. The term “homogeneous,” refers to a population that contains more than approximately 40%, 50%, 60%, 70%, 80%, 85%, 86%, 87%, 88%, 89%, 90%, 91%, 92%, 93%, 94%, 95%, 96%, 97%, 98%, or 99% of the desired cell lineage.

In one embodiment, the cell culture environment comprises a suspension culture. As used herein, the term “suspension culture” refers to a cell culture system whereby cells are not tightly attached to a solid surface when they are cultured. Non-limiting examples of suspension cultures include agarose suspension cultures, and hanging drop suspension cultures. In one embodiment, the cell culture environment comprises a suspension culture where the tissue culture medium is Dulbecco's Modified Eagle's Medium and Ham's F12 media (DMEM/F12), and it is supplemented with a fibroblast growth factor (FGF) such as FGF-2. In one embodiment, the cell culture environment comprises an FGF. In another embodiment, the cell culture environment comprises a suspension culture where the tissue culture medium is DMEM/F12, FGF-2, and MEDII conditioned medium. MEDII conditioned medium is defined in WO 99/53021, hereby incorporated by reference in its entirety. In still another embodiment, the suspension culture is an agarose suspension culture. In certain other embodiments, the cell culture environment is essentially free of human leukemia inhibitory factor (hLIF).

In other embodiments, the cell culture environment can also contain supplements such as L-Glutamine, NEAA (non-essential amino acids), P/S (penicillin/streptomycin), N2 supplement (5 g/ml insulin, 100 g/ml transferrin, 20 nM progesterone, 30 nM selenium, 100 M putrescine (Bottenstein, & Sato, 1979 PNAS USA 76:514-517) and β-mercaptoethanol (β-ME). It is contemplated that additional factors may be added to the cell culture environment, including, but not limited to fibronectin, laminin, heparin, heparin sulfate, retinoic acid, members of the epidermal growth factor family (EGFs), members of the fibroblast growth factor family (FGFs) including FGF2 and/or FGF8, members of the platelet derived growth factor family (PDGFs), transforming growth factor (TGF)/bone morphogenetic protein (BMP)/growth and differentiation factor (GDF) factor family antagonists including but not limited to noggin, follistatin, chordin, gremlin, cerberus/DAN family proteins, ventropin, and amnionless. TGF, BMP, and GDF antagonists could also be added in the form of TGF, BMP, and GDF receptor-Fc chimeras. Other factors that may be added include molecules that can activate or inactivate signaling through Notch receptor family, including but not limited to proteins of the Delta-like and Jagged families as well as gamma secretase inhibitors and other inhibitors of Notch processing or cleavage. Other growth factors may include members of the insulin like growth factor family (IGF), the wingless related (WNT) factor family, and the hedgehog factor family. Additional factors may be added to promote neural stem/progenitor proliferation and survival as well as neuron survival and differentiation. These neurotrophic factors include but are not limited to nerve growth factor (NGF), brain derived neurotrophic factor (BDNF), neurotrophin-3 (NT-3), neurotrophin-4/5 (NT-4/5), interleukin-6 (IL-6), ciliary neurotrophic factor (CNTF), leukemia inhibitory factor (LIF), cardiotrophin, members of the transforming growth factor (TGF)/bone morphogenetic protein (BMP)/growth and differentiation factor (GDF) family, the glial derived neurotrophic factor (GDNF) family including but not limited to neurturin, neublastin/artemin, and persephin and factors related to and including hepatocyte growth factor. Neural cultures that are terminally differentiated to form post-mitotic neurons may also contain a mitotic inhibitor or mixture of mitotic inhibitors including but not limited to 5-fluoro 2′-deoxyuridine and cytosine-D-arabino-furanoside (Ara-C). The cell culture environment can further comprise conditions that are known to lead to an increase in endogenous ceramide levels, including but not limited to ionizing radiation, UV light radiation, application of retinoic acid, heat shock, chemotherapeutic agents such as but not limited to daunorubicin, and oxidative stress.

In other embodiments, the cell culture environment comprises seeding the cells or an embryoid body to an adherent culture. As used herein, the terms “seeded” and “seeding” refer to any process that allows an embryoid body or a portion of an embryoid body or a cell to be grown in adherent culture. An used herein, the term “a portion” refers to at least one cell from an embryoid body, between approximately 1-10 cells, between approximately 10-100 cells from an embryoid body, or between approximately 50-1000 cells from an embryoid body. As used herein, the term “adherent culture” refers to a cell culture system whereby cells are cultured on a solid surface, which may in turn be coated with a substrate. The cells may or may not tightly adhere to the solid surface or to the substrate. The substrate for the adherent culture may further comprise any one or combination of polyornithine, laminin, poly-lysine, purified collagen, gelatin, extracellular matrix, fibronectin, tenascin, vitronectin, poly glycolytic acid (PGA), poly lactic acid (PLA), poly lactic-glycolic acid (PLGA), mouse serum, human serum, and feeder cell layers such as, but not limited to, primary astrocytes, astrocyte cell lines, glial cell lines, bone marrow stromal cells, primary fibroblasts or fibroblast cells lines, or any other chemical or biological material that allows the cells to proliferate or be stabilized in culture. In one embodiment, the substrate for the adherent culture may comprise the extracellular matrix laid down by a feeder cell layer, or laid down by the pluripotent human cell or cell culture.

As used herein, the term “enriched” refers to a culture that contains more than 50%, 60%, 70%, 80%, 90%, or 95% of the desired cell lineage.

As used herein, the term “differentiate” refers to the production of a cell type that is more differentiated than the cell type from which it is derived. The term therefore encompasses cell types that are partially and terminally differentiated.

As used herein, “serum containing” refers to a medium that contains serum or serum replacement. For example, the medium can contain greater than 1, 2, 3, 4, 5, 6, 7, 8, 9, or 10% serum or serum replacement. In certain embodiments of the invention, the pluripotent cells are exposed to a medium that contains serum or a serum replacement.

As used herein, “essentially LIF free” refers to a medium that does not contain leukemia inhibitory factor (LIF), or that contains essentially no LIF. As used herein, “essentially” means that a de minimus or reduced amount of a component, such as LIF, may be present that does not eliminate the improved cell maintenance capacity of the medium or environment. For example, essentially LIF free medium or environment can contain less than 100, 75, 50, 40, 30, 10, 5, 4, 3, 2, or 1 ng/ml LIF, wherein the presently improved cell maintenance capacity of the medium or environment is still observed.

It is contemplated that the pluripotent cells can be passaged using enzymatic, non-enzymatic, or manual dissociation methods. Non-limiting examples of enzymatic dissociation methods include the use of proteases such as trypsin, collagenase, dispase, trypLE, and accutase. In one embodiment, accutase is used to passage the contacted cells. When enzymatic passaging methods are used, the resultant culture can comprise a mixture of singlets, doublets, triplets, and clumps of cells that vary in size depending on the enzyme used. A non-limiting example of a non-enzymatic dissociation method is a cell dispersal buffer. Manual passaging techniques have been well described in the art, such as in Schulz et al., 2004 Stem Cells, 22(7):1218-38. The choice of passaging method is influenced by the choice of extracellular matrix, and is easily determined by one of ordinary skill in the art.

As used herein, the term “protease passaged” cell refers to a cell that has been passaged using a protease treatment such that the cells were cultured as an essentially single cell culture. In one embodiment, the protease treatment comprises the sequential use of collagenase and trypsin, however, other protease treatments known now or later developed are encompassed within the term.

The cell types that are stabilized using the techniques of the current invention have several uses in various fields of research and development including but not limited to drug discovery, drug development and testing, toxicology, production of cells for therapeutic purposes as well as basic science research. These cells can be differentiated to cell types that express molecules that are of interest in a wide range of research fields. These include the molecules known to be required for the functioning of the various cell types as described in standard reference texts. These molecules include, but are not limited to, cytokines, growth factors, cytokine receptors, extracellular matrix, transcription factors, secreted polypeptides and other molecules, and growth factor receptors.

Human pluripotent cells offer unique opportunities for investigating early stages of human development as well as for therapeutic intervention in several disease states, such as diabetes mellitus and Parkinson's disease, such as by the development of pure tissue or cell types. For example, the use of insulin-producing β-cells derived from hESCs would offer a vast improvement over current cell therapy procedures that utilize cells from donor pancreases. Currently cell therapy treatments for diabetes mellitus, which utilize cells from donor pancreases, are limited by the scarcity of high quality islet cells needed for transplant. Cell therapy for a single Type I diabetic patient requires a transplant of approximately 8×10⁸ pancreatic islet cells (Shapiro et al., (2000) N Engl J. Med., 343:230-238; Shapiro et al., (2001) Best Pract Res Clin Endocrinol Metab., 15:241-264; Shapiro et al., (2001) Bmj., 322:861). As such, at least two healthy donor organs are required for to obtain sufficient islet cells for a successful transplant. HESCs offer a source of starting material from which to develop substantial quantities of high quality differentiated cells for human cell therapies.

Throughout this application, various publications are referenced. The disclosures of all of these publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments.

### EXAMPLES

### Experimental Procedures

### Mouse Cell Culture and Transfection

D3 (Doetschman et al., (1985) J. Embryol. Exp. Morph., 87:27-45) and R1-EGFP (Hadjantonakis et al., (1998) Mech. Dev., 76:79-90) mouse ES cells were cultured on gelatinized plates (0.2% gelatin in PBS) in ES complete (ESC) medium consisting of high glucose DMEM supplemented with 10% FCS, 1 mM L-glutamine, 100 U/ml penicillin, 100 U/ml streptomycin, 1 mM sodium pyruvate, 0.1 mM β-mercaptoethanol, 10% Knockout Serum Replacer (KSR; Invitrogen) at 37° C. under 10% CO₂, supplemented with 1×10³ units/ml LIF (ESGRO, Chemicon). Alternatively, cells were grown in ESC media conditioned by L-M tk-cells (ATCC, CRL-2648) or by Wnt3a secreting L-M tk-cells (ATCC, CRL-2647), prepared as per the suppliers recommendations (see Sato et al., (2004) Nature Med., 10:55-63). Cells were passaged every 3 days using trypsin-EDTA (Invitrogen) and plated at 1.6×10⁵ cells/cm². For the generation of stable transfectants, 2×10⁷ cells were resuspended in 1 ml PBS and electroporated with 20 μg linearized plasmid DNA (200 V, 500 μF). After 24 hours recovery, transfected cells were selected in puromycin (1 μg/ml) or neomycin (200 μg/ml) for between 7-10 days then clonally expanded in the presence of drug selection. Four independent clonal cell lines were typically selected for analysis to determine that the properties of each cell line set were consistent.

### Routine Human ES Cell Culture

The human embryonic stem cell line BG01 (BresaGen, Inc., Athens, Ga.) was used in this work. BG01 cells were grown in hES Medium, consisting of DMEM/F-12 (50/50) supplemented with 20% knockout serum replacer (KSR; Invitrogen), 0.1 mM MEM Non-essential amino acids (NEAA; Invitrogen), 2 mM L-Glutamine (Invitrogen), 50 U/ml penicillin, 50 μg/ml streptomycin (Invitrogen), 4 ng/ml bFGF (Sigma) and 0.1 mM β-mercaptoethanol (Sigma). The cells were grown on mouse primary embryonic fibroblast feeder layers that were mitotically inactivated with mitomycin C. Feeder cells were plated at 1.2×10⁶ cells per 35 mm dish. BG01 cells were passaged using a collagenase/trypsin method. Briefly, medium was removed from the dish, 1 ml of 200 U/ml Collagenase type IV (GibcoBRL) was added, and the cells were incubated at 37° C. for 1-2 minutes. Collagenase was removed and 1 ml of 0.05% trypsin/0.53 mM EDTA (GIBCO) was applied. Cells were incubated at 37° C. for 30 seconds and then washed from the feeder layer, and the trypsin was inactivated in DMEM/F-12 with 10% fetal bovine serum (FBS; Hyclone). Cells were replated on feeder layers at 100,000-300,000 cells per 35 mm dish and were passaged every 3 days.

### Growth of BG01 Cells in Feeder Free Conditions

hES medium (25 mls) was conditioned overnight on mitomycin treated MEFs plated in 75 cm² flasks at 56,000 cells/cm². The MEFs were used for up to 1 week with conditioned medium (CM) collection every 24 hours. CM was supplemented with an additional 8 ng/ml of hbFGF before use (MEF-CM). Matrigel coated dishes were prepared by diluting Growth Factor Reduced BD matrigel matrix (BD Biosciences) to a final concentration of 1:30 in cold DMEM/F-12. 1 ml/35 mm dish was used to coat dishes for 1-2 hours at room temperature or at least overnight at 4° C. Plates were stored up to one week at 4° C. Matrigel solution was removed immediately before use.

ES cell maintenance, differentiation assays, LIF titration assays and flow cytometry

For long-term maintenance, mycER and myc^{T58A}ER mouse ES cells were grown in ES complete media supplemented with 4-hydroxytomoxifen (4OHT) (100 nM or 2-10 nM, respectively) and re-fed every second day. For differentiation of ES cells as embryoid bodies (EB), cells were trypsinized and replated on bacteriological dishes at 1.6×10⁵ cells/cm² and grown in ESC media (−LIF) plus or minus 4OHT to allow for EB formation. Cells were re-fed every second day and expanded after 4 days of culture. mycER cells were passaged by limited trypsinization, avoiding generation of single cell suspensions. AP activity was assayed with Alkaline Phosphatase Substrate Kit I (Vector Laboratories) according to the manufacturers' instructions. LIF titration assays were performed over a range of 0 to 1×10³ units ESGRO per ml in the presence or absence of 4OHT by plating cells in 24 well plates. For flow cytometry, ES cells (2×10⁶) were washed with 1×PBS and fixed in 2% paraformaldehyde in 1×PBS for 10 minutes at room temperature. Cells were then washed (1×PBS), incubated with anti-SSEA1 mouse monoclonal antibody (Chemicon, 1:100) at 4° C. for 30 minutes, washed twice and resuspended in anti-mouse Alexa-488 secondary antibody (1:1,000; Molecular Probes) in 1% BSA/PBS at 4° C. for 30 minutes. Finally, cells were washed twice, resuspended in 1% BSA/1×PBS and analysis performed using a Beckman Coulter FC500 flow cytometer.

### Plasmid Construction

To generate pCAGIneo, a Kpn I-Bam HI fragment from pEFIRESneo (Hobbs et al., (1998) Biochem. Biophys. Res. Comm., 252:368-372) was inserted into Kpn I-Bam HI cut pCAGIpuro (Pratt et al., (2000) Dev. Biol., 228:19-28). An Eco RI fragment containing a human mycER fusion from pBabeMycER (a gift from K. Helin) was used subcloned into pCAGIpuro or pCAGIneo to generate pCAGIMycERpuro/neo. To generate the myc^{T58}ER construct, mycER was subcloned into pBSK (Stratagene) as an EcoRI fragment and used as a template for Quickchange mutagenesis (Stratagene). The primers used to generate the threonine to alanine mutation at amino acid 58 were:

(SEQ ID NO:1)

5′-TTCGAGCTGCTGCCCGCCCCGCCCCTGTCCCCTAGC-3′
and

(SEQ ID NO:2)

5′-GCTAGGGGACAGGGGCGGGGCGGGCAGCAGCTCGAA-3′.

The mutated construct was sequenced and re-cloned as an Eco RI fragment into pCAGIpuro or pCAGIneo. A dominant negative version of mycER, myc^{Δ40-178}ER, lacking part of the c-myc transactivation domain (amino acids 40-178) was generated by excising a Pst I fragment in the open reading frame and recircularizing (Hermeking et al., (1994) Proc. Natl. Acad. Sci. USA, 91:10412-10416). To generate an EF1α-driven, FLAG-tagged STAT3 expression construct, a Nhe I-Not I STAT³FLAG PCR fragment, using pS3FLAG (Horvath et al., (1995) Genes Dev., 9:984-994) as a template, was inserted into pEFIRESNeo (Hobbs et al., (1998) Biochem. Biophys. Res. Comm., 252:368-372).

Human ES cells and mouse ES cells were transfected with the mycER and myc^{T58A}ER plasmids. Human ES cells were plated on Matrigel in 6-well trays as described above, and transfected with the plasmids using Lipofectamine 2000 (Invitrogen), according to the manufacturer's instructions. Transfection efficiency was monitored by co-transfection with a FITC-labeled oligonucleotide (Invitrogen, #44-2926). Transfected cells were selected 24 hours after transfection using 1 μg/ml puromycin. Typically, >80% transfection efficiency was achieved.

### Antibodies and Immunoblot Analysis

Cells were lysed by incubating for one hour on ice in lysis buffer consisting of 50 mM Hepes pH 7.5, 1 mM EDTA, 1 mM EGTA, 250 mM NaCl, 10% glycerol, 0.1% Tween-20, 10 μg/ml TPCK, 50 μg/ml TLCK, 170 μg/ml PMSF, 0.5 mg/ml leupeptin, 0.7 mg/ml pepstatin and phosphatase inhibitor cocktail II (Calbiochem) with occasional vortexing/pipetting. Whole cell extracts (20-40 μg total protein) were separated by 10% SDS-PAGE, transferred to nitrocellulose membranes (BioRad), blocked with 3% BSA in TBSE (Tris HCl pH 7.5, 150 mM NaCl, 2 mM EDTA and 0.1% NP40) and probed with the indicated antibodies at 4° C. The following antibodies were used: anti-c-myc (N-262; Santa Cruz), anti-Oct4 (N-19; Santa Cruz), anti-tubulin (Serotech), anti-HDAC1 (Zymed), anti-Cdk2 (M2; Santa Cruz), anti-GSK3β (Transduction Laboratories). The anti-HDAC1 antibody detects both HDAC1 and HDAC2 and accordingly, a doublet is seen by immunoblot analysis. An antibody that recognizes the T58 phosphorylated form of c-myc (T58/S62, Cell Signaling Technology) was used to evaluate the status of c-myc^{T58}. For protein stability assays, MG132 (5 μM, Sigma) was added to the culture media for up to 3 hours prior to harvesting cells for protein analysis.

### GSK3β Kinase Assays

Cell lysate (300 μg total protein) was adjusted to 1 mg/ml with cell lysis buffer and tumbled with 3 μg of primary antibody for 3 hours at 4° C. 30 μL of protein A Sepharose beads were added and tumbled for a further 1 hour at 4° C. The beads were washed three times with 1 ml of cold lysis buffer and once with 1 ml of kinase buffer (50 mM Hepes pH 7.5, 10 mM MgCl2, 1 mM DTT, 2.5 mM EGTA and protease inhibitor cocktail II (Calbiochem)). Immune complexes bound to the PAS beads were resuspended in 30 μL of kinase buffer with or without the addition of GSK3β inhibitor II (3 μM in ethanol, Calbiochem) or ethanol and incubated at 30° C. for 20 minutes. 10 μCi of [γ-³²P] ATP and 5 μg of myelin basic protein (MBP, Upstate Biotech) was added and the reactions incubated at 30° C. for a further 20 minutes. Reactions were terminated by the addition of 2×SDS load buffer, heated at 95° C. for 2 minutes and resolved on a 12% SDS-polyacrylamide gel. Kinase activities were quantitated by phosphorimager analysis.

### Northern Blot, RT-PCR Analysis and ChIP Assays

Total RNA from ES cells was prepared with TRIzol Reagent (InVitrogen) as per the manufacturer's instructions. Northern blot analysis and probe synthesis were performed as described previously (Stead et al., (2002) Oncogene, 21; 8320-8333; Faast et al., (2004) Oncogene, 23:491-502). First strand cDNA was generated using a SuperScript first strand synthesis kit (InVitrogen) as per the manufacturer's instructions. Primer sequences for RT-PCR analysis and PCR conditions were as described previously (Oka et al., (2002) Mol. Biol. Cell., 13:1274-1281) except for c-myc; F5′-CTCAACGACAGCAGCTCGCCC-3′ (SEQ ID NO:3), R5′-GGAGACGTGGCACCTCTTGAG-3′ (SEQ ID NO:4).

Chromatin immunoprecipitation assays were performed essentially as described by Weinmann et al., ((2001) Mol. Cell. Biol., 21:6820-6832) using primers directed against the mouse c-myc and CDK1 promoters. Primer sequences were as follows: c-myc-F, 5′-TCCTCTTTCCCCGGCTCCCCACTAG-3′ (SEQ ID NO:5), c-myc-R, 5′-GCAAAGCCCCTCTCACTCCAGAGCT-3′ (SEQ ID NO:6), CDK1-F, 5′-GGTAAAGCTCCCGGGATCCGCCAAT-3′ (SEQ ID NO:7), and CDK1-R: 5′-GTGGACTGTCACTTTGGTGGCTGGC-3′ (SEQ ID NO:8).

### Generation and Analysis of Chimeric Mice

4OHT-maintained R1-EGFP/myc^{T58A}ER ES cells were injected into blastocyst stage C57BL/6 embryos and reimplanted into pseudopregnant females as described in Hogan et al., (1994) Manipulating the Mouse Embryo: A Laboratory Manual. 2nd edition. (Cold Spring Harbor Press).

### Q-PCR Gene Expression Assay

Q-PCT was performed using relative quantitation using SYBR Green chemistry on the Rotor Gene 3000 instrument (Corbett Research) and a two-step RT-PCR format. Primers were designed to lie over exon-exon boundaries or span introns of at least 800 bp when possible, as this has been empirically determined to eliminate amplification from contaminating genomic DNA. When marker genes were employed that do not contain introns or they possess pseudogenes, DNase I treatment of RNA samples was performed. The human specificity of these primer sets has also been demonstrated.

Total RNA was isolated using RNeasy (Qiagen) and quantitated using RiboGreen (Molecular Probes). Reverse transcription from 350-500 ng of total RNA was carried out using the iScript reverse transcriptase kit (BioRad), which contains a mix of oligo-dT and random primers. Each 20 μL reaction was subsequently diluted up to 100 μL total volume and 3 μL was used in each 10 μL Q-PCR reaction containing 400 nM forward and reverse primers and 5 μL 2×SYBR Green master mix (Qiagen). Two step cycling parameters were used employing a 5 second denature at 85-94° C. (specifically selected according to the melting temp of the amplicon for each primer set) followed by a 45 second anneal/extend at 60° C. Fluorescence data was collected during the last 15 seconds of each extension phase. A three point, 10-fold dilution series was used to generate the standard curve for each run and cycle thresholds (Ct's) were converted to quantitative values based on this standard curve. The quantitated values for each sample were normalized to housekeeping gene performance and then average and standard deviations were calculated for triplicate samples. At the conclusion of PCR cycling, a melt curve analysis was performed to ascertain the specificity of the reaction. A single specific product was indicated by a single peak at the Tₘ appropriate for that PCR amplicon. In addition, reactions performed without reverse transcriptase served as the negative control and do not amplify.

Both Cyclophilin G and GUS were used to calculate a normalization factor for all samples. The use of multiple HGs simultaneously reduces the variability inherent to the normalization process and increases the reliability of the relative gene expression values (Vandesompele et al., 2002, Genome Biol., 3:RESEARCH0034).

Transfection and Knockdown of GSK3β with RNAi

BG01 cells were plated as described above on Matrigel in 6-well plates. GSK3β RNAi duplexes (100 nM, Validated RNAi DuoPack; #45-1488, Invitrogen) and a negative control RNAi (#46-2001) were separately transfected into the hESCs with Lipofectamine 2000 (Invitrogen). Transfections were performed according to the manufacturer's instructions. Transfection efficiency was monitored by co-transfection with a FITC-labeled oligonucleotide (Invitrogen, #44-2926). Typically, >80% transfection efficiency was achieved.

### Example 1

### Sustained c-myc Expression Severely Delayed Mouse ES Cell Differentiation

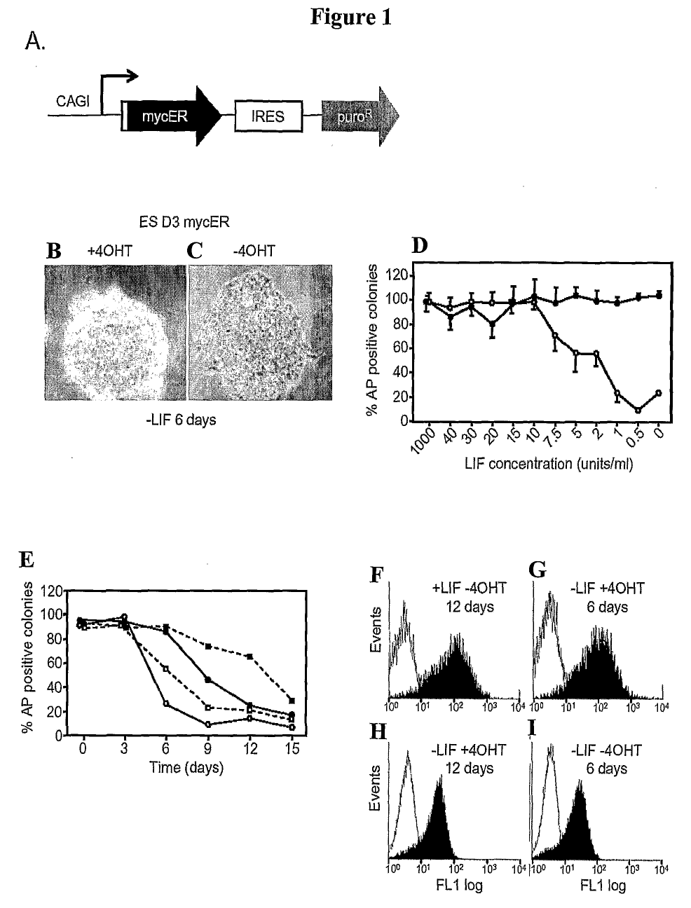

To identify downstream effectors of LIF-STAT3 signaling, a panel of genes that were known to be regulated by STAT3 in other contexts were screened for their ability to support self-renewal under conditions where their expression was enforced. This panel included genes involved in cell cycle progression such as c-fos, jun B, cyclin D1 and c-myc in addition to regulators of apoptosis such as bcl-2 and Pim-1 (see Hirano et al., (2000) Oncogene, 19:2548-2556; Zhang et al., (2003) Oncogene, 22:894-905). Candidate cDNAs were expressed constitutively from the human EF1α promoter (Hobbs et al., (1998) Biochem. Biophys. Res. Comm., 252:368-372), the CAGI promoter (Pratt et al., (2000) Dev. Biol., 228:19-28) or, from the CAGI promoter as a fusion protein linked to the steroid binding domain of the estrogen receptor (FIG. 1A). By addition of 4OHT to cultures, the labile cytoplasmic form of ER fusions can be switched to a biologically active state (Eilers et al., (1989) Nature, 340:66-68; Pelengaris et al., (1999) Mol. Cell, 3:565-577). In the case of nuclear factors, this involves their steroid-induced translocation to the nucleus. The initial screen involved transfection of expression constructs, the selection of puromycin-resistant ES cell colonies in the presence of LIF and the passaging of clonal cell lines in the absence of LIF for a further 6 days. In the case of CAGI-ER cell lines, this involved parallel cultures where 4OHT was included or omitted. Cell lines were then evaluated by morphological criteria, together with a determination of SSEA1 status and alkaline phosphatase (AP) activity. The only STAT3 target gene that maintained a uniform, domed shaped ES cell colony morphology with robust AP activity and high SSEA1 levels in this assay was c-myc (FIG. 1 and data not shown). Similar results were obtained in the EF1α, the CAGI and CAGI-ER expression systems (data not shown) but because of the advantages associated with an inducible system, all further experiments were restricted to the analysis of CAGI-ER cell lines.

In the presence of 4OHT (mycER on), ES cells retained a uniform dome-shaped colony morphology (FIG. 1B), similar to that of LIF maintained cells (data not shown). In contrast, withdrawal of 4OHT promoted a distinct flattening of colonies with a less uniform appearance (FIG. 1C) that correlated with ES cells undergoing differentiation in the absence of LIF (data not shown). The ability of c-myc to promote ES cell self-renewal was confirmed in a LIF titration assay where mycER ES cells were grown in the presence or absence of 4OHT for 6 days in media containing LIF over a range of 0-1×10³ units per ml and then assayed for AP activity. Over the entire LIF titration range, mycER cells grown in 4OHT maintained AP activity (>90% positive), whereas a severe reduction (˜20% positive) was seen in the absence of 4OHT under conditions of 10 units/ml LIF or below (FIG. 1D). These results clearly demonstrated that sustained c-myc activity maintained AP activity in ES cells, under conditions where significant loss of AP activity is normally observed. MycER cells were then grown in the presence or absence of 4OHT in the absence of LIF for up to 15 days and assayed for AP activity. While mycER maintained AP activity for the first 6 days, this decreased markedly over days 6-15 indicating that mycER was delaying differentiation rather than blocking it indefinitely (FIG. 1E). Including low levels of LIF (7.5 units/ml), that by itself had only a small effect on maintaining AP levels over 15 days, appeared to further delay the loss of AP activity in mycER cells grown with 4OHT. These data indicate that LIF can cooperate with c-myc to maintain AP activity, thereby further delaying aspects of ES cell differentiation.

As an alternate assay to evaluate the ability of mycER to maintain ES cells in the absence of LIF, SSEA1 reactivity on the surface of ES cells was determined by flow cytometry. While mycER maintained SSEA1 reactivity for up to 6 days, by 12 days in the absence of LIF this was reduced to levels comparable to that in ES cells grown without 4OHT and LIF (FIGS. 1F-I). The decline in SSEA1 reactivity between days 6 and 12 was accompanied by general colony flattening and increased heterogeneity in cell morphology, consistent with differentiation occurring (data not shown). Under the conditions of these experiments, c-myc imposes a significant differentiation delay but does not maintain self-renewal indefinitely in the absence of LIF signaling.

### Example 2

### c-myc was Elevated and has Unusual Stability in Murine ES Cells

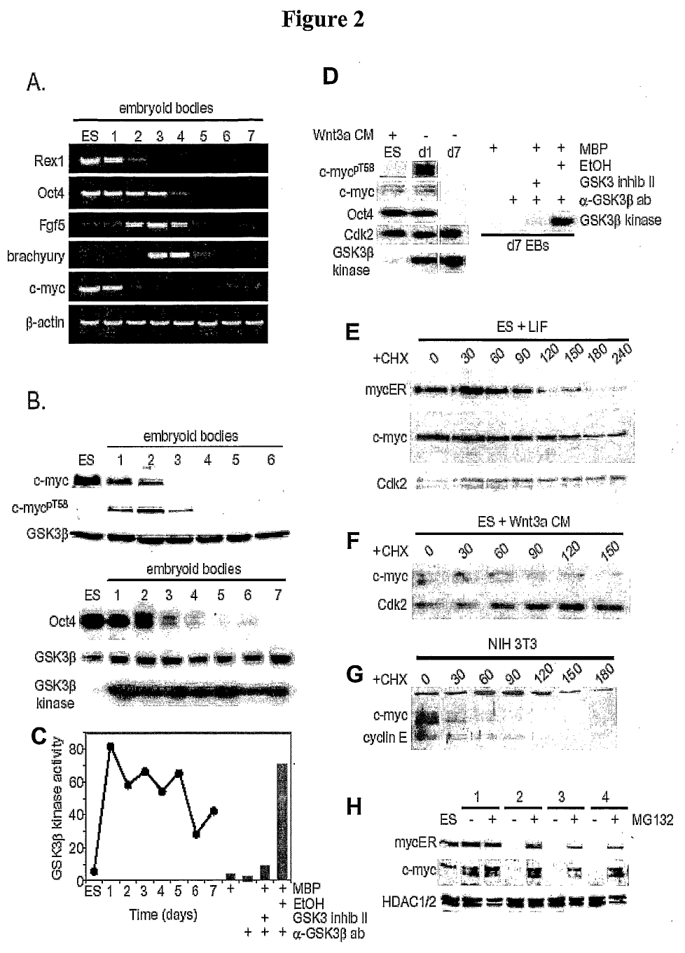

To understand more about the possible role of c-myc in ES cell self-renewal and differentiation, the regulation of c-myc in ES cells and during embryoid body (EB) differentiation was characterized. For c-myc to be a bone fide regulator of self-renewal and pluripotency in ES cells, it was predicted that its activity would be elevated in ES cells, but rapidly downregulated during differentiation. RT-PCR analysis showed that c-myc transcripts were elevated in ES cells but declined by day 2 of EB differentiation (FIG. 2B), closely paralleling the decline in Rex1 mRNA (FIG. 2A). Levels of c-myc protein were also elevated in LIF-maintained ES cells but declined markedly by day 1 of LIF withdrawal and even further over days 1-3 (FIG. 2C). The down-regulation of c-myc protein and mRNA therefore occurs well before Oct4 levels are extinguished and prior to the appearance of early differentiation markers such as Fgf5 (early primitive ectoderm) and brachyury mRNAs (nascent mesoderm; FIG. 2A). These results indicate that elevated c-myc levels are closely associated with the ES cell state and are consistent with the hypothesis that c-myc performs a role in self-renewal.

It was frequently observed that c-myc protein levels declined slightly before the onset of c-myc mRNA downregulation following LIF withdrawal, suggesting that post-transcriptional mechanisms played a role in determining c-myc levels. A common mechanism that triggers c-myc degradation is through its phosphorylation at threonine 58 (T58) by GSK3β (Hoang et al., (1995) Mol. Cell. Biol., 15:4031-4042; Sears et al., (2000) Genes Dev., 14:2501-2514; Gregory et al., (2003) J. Biol. Chem., 278:51606-51612). To determine if T58 was phosphorylated in a manner consistent with it being targeted to initiate c-myc proteolysis following removal of LIF, the same extracts were probed with a T58 phospho-specific antibody (FIG. 2B). The pT58 form of c-myc was absent in ES cells but increased markedly upon LIF withdrawal with kinetics, consistent with it being an initiating event in the degradation of c-myc (FIG. 2B). Since GSK3β is the principal regulator of T58 phosphorylation, it was tested to determine if it could account for phosphorylation of c-myc. This was done by immunoprecipitating GSK3β from whole cell lysates and then evaluating kinase activity through its ability to phosphorylate myelin basic protein (MBP) in vitro. In ES cells, GSK3β kinase activity was low but within 1 day following LIF withdrawal was induced by approximately 15-fold (FIG. 2C). This could not be accounted for by changes in GSK3β protein levels as they remained relatively constant throughout this and other differentiation experiments (FIG. 2B). The specificity of the assay was confirmed by demonstrating that the immunoprecipitated kinase activity from day 6 EBs was sensitive to a specific inhibitor of GSK3β (FIG. 2C). Changes in GSK3β activity did not correlate with the decline in Oct4 protein levels that occurred from day 2 onwards, but paralleled closely with phosphorylation of c-myc on T58. These results support the hypothesis that GSK3 can trigger c-myc degradation through phosphorylation of T58.

Since Wnt3a activity can also maintain self-renewal and pluripotency of ES cells (Sato et al., (2004) Nature Med., 10:55-63), it was determined whether c-myc was elevated under conditions of Wnt3a dependent maintenance, and whether c-myc was phosphorylated on T58 following withdrawal of the Wnt3a signal. To test this idea L-fibroblast conditioned media (CM) was used that contained secreted Wnt3a as a source of Wnt (see Sato et al., (2004) Nature Med., 10:55-63). Wnt3a CM supports self-renewal indefinitely in the absence of LIF, in contrast to CM from a non-secreting L-fibroblast cell line that has no maintenance activity. Bioactivity of Wnt3a was confirmed by showing that it significantly upregulates intracellular β-catenin levels (data not shown). As was observed previously (FIG. 2B), c-myc was elevated and unphosphorylated on T58 but moreover, GSK3β kinase activity was low (FIG. 2D). By day 1 following Wnt3a withdrawal however, phosphorylation of c-myc on T58 and GSK3β kinase activity were markedly elevated (FIG. 2D). Activation of GSK3β and phosphorylation of T58 both occur well before Oct4 downregulation consistent with earlier observations in LIF maintained ES cells. The increase in GSK3β kinase activity is therefore consistent with it participating in T58 phosphorylation, resulting in c-myc degradation following Wnt withdrawal. These events are indistinguishable from those that occur following LIF withdrawal (see FIGS. 2B-C).

Since phosphorylation at T58 could not be detected in ES cell lysates, this suggested that signals involved in c-myc degradation, such as that generated by GSK3β, were suppressed in ES cells. If so, it was predicted that c-myc would have enhanced stability. To test this possibility, the stability of c-myc was determined in undifferentiated ES cells relative to other cell types where its stability is in the order of 30 minutes or less (Hann & Eisenman, (1984) Mol. Cell. Biol., 4:2486-2497). Cycloheximide chase experiments in ES cells showed that in the presence of LIF or Wnt3a, c-myc has a half life (t_{1/2})˜90-120 minutes in comparison to its stability in NIH 3T3 fibroblasts where the t_{1/2} is less than 30 minutes (FIGS. 2E-G). The rapid decline in c-myc levels following LIF withdrawal can therefore be explained, in part at least, by dramatic changes in its stability. The stability of the mycER fusion protein was similar to that of endogenous c-myc (FIG. 2E). The rapid turnover of c-myc protein following LIF withdrawal was blocked by the addition of MG132 confirming that c-myc degradation is proteosome dependent during EB differentiation (FIG. 2H). Recovery of detectable c-myc in day 2, 3, or 4 EBs treated with MG132 indicates that residual c-myc transcription generates a labile pool of c-myc protein that is rapidly degraded and not normally detectable by immunoblot analysis. Hence, even in the presence of residual transcription, c-myc levels are beyond the levels of detection in this assay due to rapid turnover in differentiating cells.

GSK3β activity and c-myc protein levels were determined in undifferentiated hESCs and in spontaneously differentiated hESCs by Western blotting. GSK3β activity was low in undifferentiated hESCs, and its activity increased during HESC differentiation. GSK3β activity was determined using a phosphorylation specific antibody. In addition, c-myc was shown to be elevated in undifferentiated hESCs in comparison to protein levels in spontaneously differentiated cells.

### Example 3

### GSK3β was Excluded from the Nucleus in ES Cells

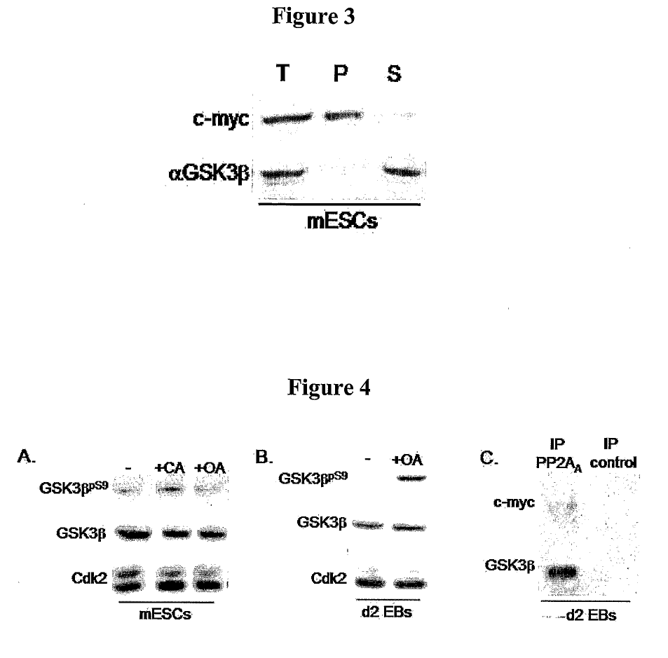

A key issue relating to the understanding of self-renewal is determining how GSK3β activity is suppressed in ESCs but activated in cells committed to differentiate. Using immunofluorescent staining, GSK3β was shown to be excluded from the nucleus in murine ESCs and human ESCs, but was also shown to localize to the nucleus in differentiated cells (data not shown). The nuclear localization of GSK3β in differentiating murine cells coincides with the degradation of c-myc, suggesting nuclear import of GSK3β is a key step in collapse of the self-renewal pathway and in the initial commitment to differentiate. The cytoplasmic localization of GSK3β in mESCs was corroborated by subcellular fractionation using hypotonic lysis (FIG. 3). These data indicate that GSK3β regulation is conserved between human and murine ESCs and that the GSK3β-c-myc dependent mechanism of self-renewal also applies to hESCs.

### Example 4

### Localization of GSK3β was Controlled by an Okadaic Acid-Sensitive Phosphatase Activity

Activation of GSK3β requires its dephosphorylation on S9. Dephosphorylation of S9 occurs either before or coincides with its nuclear import, suggesting that regulation of this residue may also influence its subcellular localization in addition to its enzymatic activity. GSK3β localization as a mechanism to restrict its access to substrates has only been proposed in the context of nuclear localization in neuroblastoma cells in response to pro-apoptotic signals (Bijur & Jope, (2001) J Biol. Chem., 276(40):37436-42). We therefore investigated whether GSK3β^{S9} was controlled through inactivation of a kinase or, by activation of a phosphatase (or both).

MESCs and d2 EBs were treated for the short-term (6 hours) with phosphatase inhibitors of varying specificities. None of the phosphatases tested had an effect on S9 phosphorylation in mESCs (FIG. 4A and data not shown). Calyculin A (CA, 2 nM) and okadaic acid (OA, 2 nM) however, significantly increased S9 phosphorylation in d2 EBs, whereas other phosphatases, such as cyclosporin A, did not increase S9 phosphorylation (FIG. 4B and data not shown).

These results indicated that a S9 kinase (possibly AKT or ILK) is active in ESCs and in d2 EBs and implies that changes in the activity of a phosphatase are important for control of S9. This mechanism accounts for the observed decline in phospho-S9 and activation of GSK3β that occurs during the early stages of differentiation. CA inhibits PP1 and PP2A in contrast to OA, which selectively targets PP2A. This implicated PP2A as a S9 phosphatase in this system. This is supported by experiments showing that PP2A complexes with GSK3 and c-myc in d2 EBs (FIG. 4C). When OA was added to EBs, GSK3β staining in the nucleus disappeared and reverted to a staining pattern previously seen in ESCs (data not shown). While it is possible that nuclear GSK3β was degraded during OA treatment and nascent GSK3β retained in the cytoplasm, it is also likely that GSK3β is transported out of the nucleus in response to OA treatment since only short incubation periods were used and GSK3β is normally a stable protein. This would be consistent with a model where GSK3β localization is controlled by the phosphorylation status of S9. It is known that PP2A dephosphorylates S62 on c-myc, resulting in its degradation (Sears, (2004) Cell Cycle, 3(9):1133-7). This suggests that phosphorylation of T58 (see Example 5) and dephosphorylation of S62 are coupled by the recruitment of PP2A to c-myc, through interaction with GSK3β.

### Example 5

### Stable c-myc was Required for Self-Renewal of ES Cells

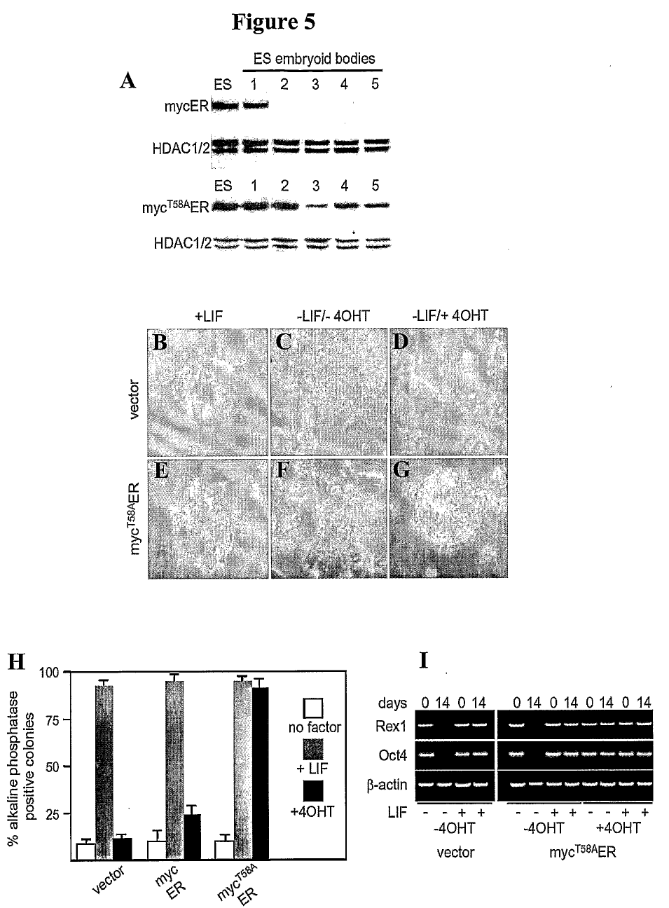

Mutation of c-myc at T58 is frequently associated with cell transformation (Pulverer et al., (1994) Oncogene, 9:59-70; Salghetti et al., (1999) EMBO J., 18:717-726), human cancer (Gregory & Hann, (2000) Mol. Cell. Biol., 20:2423-2435) and oncogenic transformation by viruses (Palmieri et al., (1983) EMBO J., 2:2385-2389) due to increased stability of the non-phosphorylatable form. To determine the contribution of T58 towards the turnover of c-myc in differentiating EBs, the stability of mycER was compared with Myc^{T58A}ER under conditions where ES cells were differentiated as EBs. Since mycER fusions were driven from a constitutive promoter (CAGI), any changes in protein levels during differentiation would likely reflect the involvement of other levels of control such as protein stability. A comparison of cell lines expressing both forms of mycER fusions revealed that the T58A mutant persisted for at least 5 days after LIF withdrawal compared to levels of the T58 form which had collapsed from day 1 (FIG. 5A). These observations confirm that T58 is a key determinant of c-myc stability in ES cells and during EB differentiation. Moreover, these results further implicate GSK3β-dependent phosphorylation of T58 as part of the mechanism that triggers c-myc turnover during differentiation.

The rapid decay of c-myc/mycER following loss of signals transduced by LIF and Wnt may explain earlier findings that enforced expression c-myc per se is not sufficient to promote self-renewal indefinitely (see FIG. 1). Under these conditions c-myc proteolysis would be accelerated, thus preventing its accumulation to levels sufficient to support long-term self-renewal. To specifically test the requirement for stable c-myc with regards to self-renewal, the ability of myc^{T58A}ER to maintain self-renewal in the absence of LIF or Wnt was evaluated in a series of experiments over 14 days (4 passages). myc^{T58A}ER cells (−4OHT) grew with properties that were indistinguishable from vector alone cell lines in the presence of LIF and maintained a typical domed-shaped ES cell structured colony consisting of tightly packed cells (FIG. 5B-G). Upon withdrawal of LIF and in the absence of 4OHT, the colony morphology of control cells (vector alone) and myc^{T58A}ER cells changed to generate a clear morphology characteristic of differentiating cells and was accompanied by loss of alkaline phosphatase (AP) activity (FIG. 5B-H). During the initial stages (days 1-4) following addition of 4OHT, myc^{T58A}ER colonies became more uniform in shape, rounded up noticeably and became loosely attached to gelatin-coated dishes. This uniform non-differentiated colony morphology could be maintained for extended periods (>30 days, see below). A small background of apoptosis was typically observed at intermediate concentrations of 4OHT (20-100 nM) that could be minimized at lower concentrations (1-10 nM) without compromising colony morphology or alkaline phosphatase activity (FIG. 5B-H). The apoptosis seen is consistent with the known pro-apoptotic effects of c-myc (Allen et al., (1997) Oncogene, 15:1133-1141; Shirogane et al., (1999) Immunity, 11:709-719). Overall, AP activity was comparable between LIF maintained cells and those grown in the presence of 4OHT (−LIF) over a 14-day period. In contrast, mycER colony morphology degenerated over 14 days (data not shown) and AP activity was significantly reduced even in the presence of 4OHT (FIG. 5H), consistent with previous results (see FIGS. 1F-I). These findings confirm earlier observations that mycER can only support self-renewal for limited periods. However, enforced expression of stable c-myc (T58A) is herein demonstrated to support self-renewal over much longer periods.

To confirm that myc^{T58A}ER maintained cells retained the molecular characteristics of ES cells, the levels of marker transcripts was evaluated (FIG. 5I). This analysis showed that myc^{T58A}ER cells could maintain elevated levels of Rex1 and Oct4 transcripts in a 4OHT-dependent manner over a 14-day period (4 passages). This indicated that the molecular signature of an ES cell was retained in cultures maintained by stable c-myc.

Addition of 4OHT per se did not promote self-renewal as vector alone cell lines were unstable in the absence of LIF. Furthermore, authentic c-myc could maintain self-renewal in a similar manner to mycER fusions when expressed constitutively, independently of 4OHT (data not shown).

The results indicate that enforced expression of c-myc^{T58A}, but not wild-type c-myc, can maintain ES cells over extended periods in the absence of LIF or Wnt. This would explain why previous attempts to identify roles for c-myc by enforced expression in ES cells have failed (Suda et al., (1988) Exp. Cell Res., 178:98-113; Benvenisty et al., (1992) Genes Dev., 6:2513-2523). While characterizing the function of c-myc in ES cells, it was discovered that its stability in the presence of LIF and Wnt is comparable to oncogenic forms of the protein associated with Burkitts lymphoma (Gregory & Hann, (2000) Mol. Cell. Biol., 20:2423-2435). Moreover, withdrawal of LIF or Wnt triggers its phosphorylation on T58 and proteosome-dependent degradation by a mechanism that is likely to involve GSK3β. It was reasoned that because of this unusual stability, enforced expression of a stable form of c-myc may be sufficient to support self-renewal over extended periods in the absence of pro-maintenance factors such as LIF or Wnt. This prediction was confirmed by expressing a mutant version of c-myc (T58A) that has enhanced stability, presumably due to its ability to evade GSK3β-dependent turnover. In contrast to wild-type c-myc, the T58A form maintained self-renewal and pluripotency over extended periods. Only modest amounts of nuclear c-myc^{T58A} were required for maintenance, eliminating the possibility that supra-physiological levels were required for its biological effect. Enhanced c-myc stability is therefore a key requirement for maintenance of ES cell self-renewal and pluripotency.

### Example 6

### Activation of GSK3β was Required for LY 294002 Induced Definitive Endoderm Formation

Treatment of human ES cells with LY 294002, a known inhibitor of PI3-kinase, and activin has been shown to induce definitive endoderm formation (data not shown; U.S. Patent Application No. 60/601,664, hereby incorporated by reference in its entirety). Akt was shown to phosphorylate and inhibit GSK3β activation, and blocking Akt promoted definitive endoderm (DE) formation (data not shown). GSK3β activation was determined to be necessary for DE formation from human ES cells.

BG01 cells were passaged on matrigel using the collagenase/trypsin method as described above and were plated on matrigel-coated chamber slides in MEF-CM. After approximately 24 hours, the medium was changed, and the cells were treated with 60 μM LY 294002, 6-bromoindirubin-3′-oxime (BIO), meBIO, or DMSO for approximately 3-4 days. The inhibitor BIO (Dr. Ali Brivaalou) was diluted in DMSO and was used at a concentration of approximately 0.1-5 μM. MeBIO (Dr. Ali Brivanlou) was also diluted in DMSO and used at a concentration of approximately 1-5 μLM. Assays were performed in triplicate and are shown as +/−SEM.

Cells were grown in these conditions for approximately 3-4 days with a medium change every 24 hours. Cells were harvested using the collagenase/trypsin method for Q-PCR as described above.

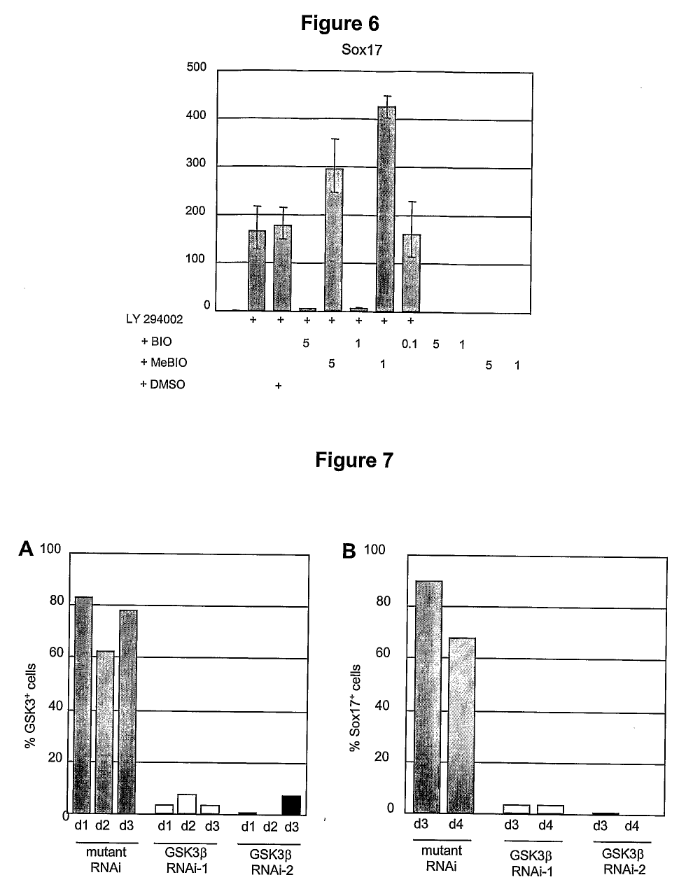

Addition of the GSK3β inhibitor, BIO, but not meBio, blocked LY 294002 induced DE formation as determined by Sox17 expression (FIG. 6).

In addition, knockdown of GSK3β expression with two specific RNAi molecules, but not a mutant RNAi, blocks the ability of LY 294002 to promote DE formation. BG01 cells were passaged from feeders using the collagenase/trypsin method and were plated on matrigel coated chamber slides in MEF-CM as described above. After approximately 24 hours, the medium was changed, and the cells were transfected with various RNAi sequences. The cells were transfected with 100 nM of the RNAi duplexes with Lipofectamine 2000 (Invitrogen). GPK3β wild-type 1 and 2 were purchased from Invitrogen (GSK3beta Validated RNAi DuoPack; #45-1488) as was the control mutant RNAi (Invitrogen, #46-2001).

12 hours after transfection, LY 294002 was added to all wells. The medium was changed daily. Cells were grown in these conditions for approximately 1-4 days, and the cells were subsequently fixed and immunostained at days 1, 2, 3, and 4 as indicated in FIGS. 7A and B.

Cells to be immunostained were rinsed once with 1×PBS and fixed for 10 minutes in 4% PFA/4% sucrose in PBS pH 7.4 at room temperature. They were then rinsed 3× in 1×PBS and blocked in 3% goat serum with 0.1% Triton-X100 in PBS for 1 hour at room temperature. Primary antibodies were diluted in 3% goat serum in PBS and this solution was applied overnight at 4° C. The primary antibodies used were pan-GSK (BD Biosciences, Cat. #610202), used at a 1:1000 dilution and rat anti-human SOX17 (obtained from CyThera, Inc.), used at 1:1000 dilution. Cells were washed for 1 hour with 3 changes of 1×PBS. Secondary antibodies were applied for 2 hours at room temperature. Secondary antibodies used were goat anti-rabbit Alexa Fluor 488 and goat anti-rat Alexa Fluor 594 (Molecular Probes), both at a 1:1000 dilution in 3% goat serum in 1×PBS. Cells were washed for 1 hour with 3 changes of 1×PBS. The chambers were removed and slides were mounted in VectaShield mounting medium with DAPI (Vector). Greater than 200 cells per sample were scored, and assays were performed in duplicate.

FIGS. 7A and B show the percentage of GSK3β and Sox17 positive cells, respectively, upon treatment with the different RNAi molecules. Treatment with the GSK3β specific RNAi sequences decreased expression of both GSK3β and Sox17, while treatment with the control RNAi sequence did not decrease expression of either GSK3β or Sox17. Therefore, GSK3β activation is necessary for the induction of DE by LY 294002, and inhibition of GSK3β inhibits differentiation of human ES cells.

These results indicate that as in mouse ES cells, GSK3β activity is also necessary to regulate the differentiation of human ES cells. Down-regulating the activation of GSK3β with a specific inhibitor or via specific RNAi molecules inhibited the formation of definitive endoderm, and strongly suggests that self-renewal of human ES cells is dependent upon inactivation of GSK3β.

Degradation of c-myc requires its phosphorylation by GSK3 β on T58, leading to its ubiquitin-dependent degradation (Hoang et al., (1995) Mol. Cell. Biol., 15:4031-4042; Sears et al., (2000) Genes Dev., 14:2501-2514; Gregory et al., (2003) J. Biol. Chem., 278:51606-51612). This is intriguing since GSK3β inhibition was recently shown to promote self-renewal of mouse and human ES cells in the absence of LIF or Wnt (Sato et al., (2004) Nature Med., 10:55-63). A role for GSK3β in promoting differentiation through T58-dependent degradation is consistent with the kinetics of GSK3β activation and c-myc T58 phosphorylation following LIF/Wnt withdrawal. These observations point towards a scenario where maintenance of ES cells requires elevated c-myc levels, achieved in part through the suppression of GSK3β activity. Activation of GSK3β following LIF/Wnt withdrawal can then account for decreased c-myc stability and loss of a stable self-renewal pathway. The findings provide a potential mechanism for how the GSK3β inhibitor BIO functions to promote self-renewal of ES cells (Sato et al., (2004) Nature Med., 10:55-63). It is predicted that suppression of GSK3β activity by BIO, in the absence of LIF/Wnt signaling, would establish conditions where c-myc was unphosphorylated on T58 leading to elevated c-myc levels and enhanced stem cell stability. This scenario is also applicable to human ES cells where BIO has a pro-maintenance function (Sato et al., (2004) Nature Med., 10:55-63).

### Example 7

### c-myc Maintained ES Cells Retain Pluripotency

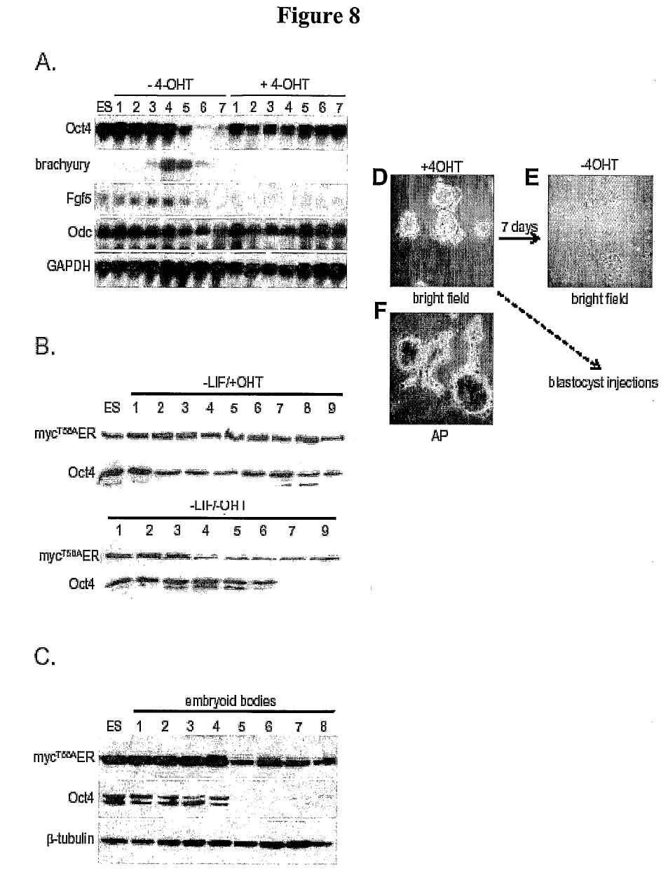

Although c-myc maintained cells retained elevated levels of ES cell specific transcripts, AP activity and SSEA1 cell surface reactivity, it was possible that the differentiation potential of these mouse cells was partially or severely restricted. To address this issue, the ability of myc^{T58A}ER cells to differentiate as EBs was tested in the absence of LIF after first being maintained for 14 days in 4OHT. Northern blot analysis showed that these cells show no obvious signs of differentiation by mRNA marker analysis when grown for a further 7 days as aggregates in the presence of 4OHT. However, EBs grown in the absence of 4OHT (FIG. 8A) differentiated, as indicated by the down-regulation of Oct4 mRNA (see Stead et al., (2002) Oncogene, 21:8320-8333; Faast et al., (2004) Oncogene, 23:491-502), the emergence of the primitive ectoderm marker Fgf5 and the nascent mesoderm marker, brachyury (FIG. 8A). Oct4 protein levels collapsed in EBs grown without 4OHT, confirming that myc^{T58A}ER cells retained the capacity to differentiate, in contrast to 4OHT maintained cells where Oct4 levels remained elevated (FIG. 8B).

The differentiation potential of cells maintained for extended periods in the presence of 4OHT (−LIF/Wnt) was then evaluated. First, EBs were generated in the absence of 4OHT from cells that had first been grown for thirty days in the presence of 4OHT (−LIF). The ability of these cells to differentiate as EBs was then determined by assessing Oct4 protein levels over a period of eight days. These cells down-regulated Oct4 at day four, indicating that they had retained the capacity to differentiate (FIG. 8C). Cells maintained for 30 days in 4OHT were confirmed to be AP positive and when plated onto gelatin coated plastic in the absence of 4OHT, did not form domed shaped colonies but spread out in a manner that was similar to spontaneously differentiating cells (FIGS. 8D-F) and furthermore, exhibited low AP activity (data not shown).

As an in vivo test of pluripotency, the ability of long-term 4OHT maintained myc^{T58A}ER ES cells to contribute to the three embryonic germ layers following injection into blastocyst stage embryos was evaluated. GFP marked R1 ES cells (Hadjantonakis et al., (1998) Mech. Dev., 76:79-90), carrying the myc^{T58A}ER transgene were generated, and were maintained in the absence of LIF (+4OHT) for 30 days (10 passages). GFP myc^{T58A}ER cells were injected into blastocyst stage C57BL/6 embryos that were then reimplanted into recipient females and then analyzed by fluorescence microscopy at 12.5 dpc. The extensive integration of GFP marked cells in all tissues was indicative that c-myc maintained ES cells contributed to all three embryonic germ layers and hence, retained pluripotency (data not shown). The relative distribution and degree of chimerism of myc^{T58A}ER cells was indistinguishable from the parental R1 GFP ES cell line (data not shown). From these analyses, it was concluded that c-myc maintains the pluripotentiality of ES cells over the time course of experiments described.

### Example 8

### c-myc was a Downstream Target of LIF/STAT3 Signaling in Mouse ES Cells

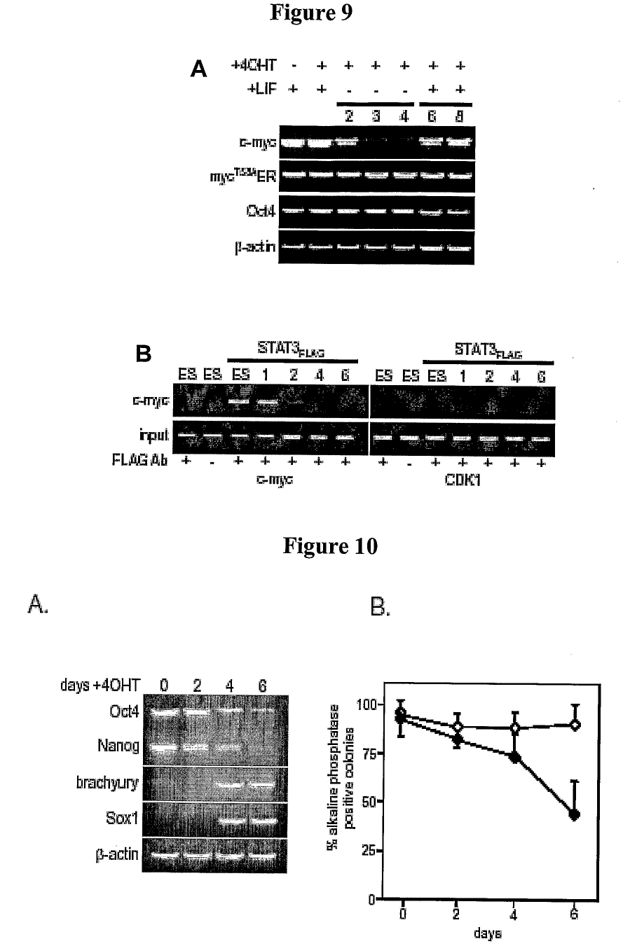

Since c-myc was able to maintain ES cells in the absence of LIF and because it is a known LIF/STAT3 target in other cell types (Kiuchi et al., (1999) J. Exp. Med., 189:63-73; Barre et al., (2003) J. Biol. Chem., 278:2990-2996; Shirogane et al., (1999) Immunity, 11:709-719; Bowman et al., (2001) Proc. Natl. Acad. Sci. USA, 98:7319-7324), it was tested whether c-myc gene activity was dependent on LIF signaling in ES cells. The ability of myc^{T58A}ER to maintain self-renewal (+4OHT) was exploited to allow for the evaluation of endogenous c-myc gene activity in the absence of LIF. Under conditions where myc^{T58A}ER maintained self-renewal independently of LIF, endogenous c-myc mRNA levels declined markedly by 3-4 days following LIF withdrawal, but were rescued by subsequent re-addition of LIF (days 6, 8; FIG. 9A). This indicates that c-myc transcription is responsive to LIF signaling in ES cells.

c-myc was determined to be part of the LIF/gp130-STAT3 signaling pathway by examining whether the endogenous c-myc gene was a direct target of STAT3 by chromatin immunoprecipitation (CHIP) analysis. FLAG epitope tagged STAT3 (STAT3_{FLAG}) was immunoprecipitated with cross-linked chromatin and its association with the c-myc promoter evaluated by PCR analysis. Specific co-precipitation between STAT3FLAG and the c-myc promoter was seen in ES cells but this decreased during EB differentiation from day 1 onwards (FIG. 9B). This is consistent with the DNA binding activity of STAT3 decreasing following cessation of LIF signaling. To confirm that STAT3 binding was specific to the c-myc gene, it was shown that STAT3 was not detected on the CDK1 promoter at any time point during the experiment. The c-myc gene therefore appears to be a direct target of LIF-STAT3 signaling through the direct action of STAT3 on the c-myc promoter.

Although ES cell self-renewal can be maintained by LIF/STAT3 and Wnt-dependent signaling or, by direct suppression of GSK3β activity, the molecular targets regulated by these pathways have not been previously defined. c-myc was identified as a LIF responsive target that was directly regulated by STAT3. These observations are consistent with reports describing transcriptional regulation of c-myc by STAT3 in response to IL-6/gp130 dependent (Kiuchi et al., (1999) J. Exp. Med., 189:63-73; Barre et al., (2003) J. Biol. Chem., 278:2990-2996) and independent pathways (Shirogane et al., (1999) Immunity, 11:709-719; Bowman et al., (2001) Proc. Natl. Acad. Sci. USA, 98:7319-7324). Enforced expression of the c-myc gene severely delayed ES cell differentiation, but could not maintain self-renewal indefinitely. While characterizing c-myc regulation in ES cells it was found that it exhibits unusual stability that changes to a more labile state with rapid turnover upon withdrawal of LIF/Wnt. Expression of a mutant form of c-myc (T58A), with comparable stability to authentic c-myc in ES cells, renders self-renewal independent of LIF or Wnt. Hence, elevated levels of stable c-myc have functional significance for the maintenance of self renewal and pluripotency. The results presented herein demonstrate that down-regulation of c-myc transcription and accelerated turnover of c-myc protein are required for commitment to differentiate. The effect c-myc has on ES cell self-renewal appears to be a general property of the myc family as N-myc is also capable of performing a similar function (data not shown).

While there was no prior evidence implicating a role for c-myc in ES cell maintenance, two reports are consistent with these findings. First, expression of an RLF/L-myc minigene, which frequently arises from a chromosomal translocation event in human small lung carcinomas (Makela et al., (1991) Mol. Cell. Biol., 11:4015-4021; Makela et al., (1991) EMBO J., 10:1331-1335; Makela et al., (1992) Oncogene, 7:405-409), delays differentiation of ES cell EBs and interferes with embryonic development (MacLean-Hunter et al., (1994) Oncogene, 9:3509-3517). Assuming that this differentiation delay was a consequence of elevated myc function, this result is consistent with the observations showing that c-myc with normal stability can delay, but not permanently block differentiation. Second, elevated c-myc activity blocks the differentiation of erythroid (Bar-Ner et al., (1992) Cell Growth Diffn., 3:183-190; Delgado et al., (1995) Oncogene, 10:1659-1665; Canelles et al., (1997) Oncogene, 14:1315-127) and myeloid (Schreiner et al., (2001) Cancer Res., 61:6480-6486; Selvakumaran et al., (1996) Blood, 4:1248-1256) lineages. Members of the myc family therefore have defined roles in controlling cell fate decisions in other biological contexts.

Gene knockout studies have so far failed to directly define a role for c-myc in development of the pluripotent embryonic epiblast (Davis et al, (1993) Genes Dev., 7:671-682; Stanton et al., (1992) Genes Dev., 6:2235-2247) probably because of functional redundancy amongst the myc family (Malynn et al., (2000) Genes Dev., 14:1390-1399). However, the obligatory binding partner of myc, Max, is essential for early embryonic growth and development (Shen-Li et al., (2000) Genes Dev., 14:17-22) and is required for all known biological functions of myc. The essential role of Max in the early embryo therefore implicates an important role for myc family members in development of the embryonic epiblast, which would be compatible with it having a role in maintenance and regulation of the pluripotent state.

Under conditions where self-renewal is dependent on LIF, the c-myc gene is under the direct control of STAT3. Upon LIF withdrawal, STAT3 DNA binding activity diminishes resulting in down regulation of c-myc transcription during the early stages of differentiation. These observations are consistent with reports that c- and N-myc transcripts are rapidly down-regulated in F9 teratocarcinoma cells upon differentiation (Jakobovits et al., (1985) Nature, 318:188-191; Griep & DeLuca, (1986) Proc. Natl. Acad. Sci. USA, 83:5539-5543; Sejersen et al., (1987) Exp. Cell. Res., 172:304-317) and that myc family members are elevated in pluripotent cells of the mouse embryo (Downs et al., (1989) Genes Dev., 3:860-869).

Based on the results presented herein, the ability of STAT3 to target c-myc would explain its ability to promote self-renewal of ES cells (Niwa et al., (1998) Genes Dev., 12:2048-2060; Ernst et al., (1999) J. Biol. Chem., 274:9729-9737; Matsuda et al., (1999) EMBO J., 18:4261-4269). In other cases where STAT3 activates c-myc, it co-regulates anti-apoptotic genes such as Pim-1/2 hence, suppressing the pro-apoptotic function of c-myc (Fukada et al., (1996) Immunity, 5:449-460; Shirogane et al., (1999) Immunity, 11:709-719). The ability of c-myc, but not STAT3, to induce cell death implies that STAT3 activates targets, in addition to c-myc, which have anti-apoptotic functions. The biological importance of this is illustrated in STAT3-dependent tumor development where oncogene cooperativity between pro-proliferative (c-myc) and anti-apoptotic (Pim-1/2) activities are crucial (Allen et al., (1997) Oncogene, 15:1133-1141; Shirogane et al., (1999) Immunity, 11:709-719; White, (2003) Genes Dev., 17:1813-1816). Additional STAT3 target genes in ES cells are identified, and anti-apoptotic pathways that work in collaboration with c-myc are defined. The control of c-myc transcription in ES cells by Wnt through a mechanism involving the β-catenin/TCF-LEF pathway is determined, as has been demonstrated in other cell types (Moon et al., (2002) Science, 296:1644-1646; van Es et al., (2003) Curr. Opin. Genet. Dev., 13:28-33).

### Example 9

### Self-Renewal of Murine ES Cells is Dependent on c-myc Activity

To demonstrate that c-myc is required for self-renewal and maintenance of ES cells, stable mouse cell lines expressing a mycER fusion where the c-myc open reading frame lacked most of its transactivation domain (myc^{Δ40-178}ER) were generated. This and similar dominant negative versions of c-myc have previously been shown to promote differentiation without necessarily imposing a cell cycle arrest (Canelles et al., (1997) Oncogene, 14:1315-127; Schreiner et al., (2001) Cancer Res., 61:6480-6486). While ES cell colonies with a typical domed-shape morphology could be maintained in the presence of LIF and in the absence of 4OHT over extended periods (>30 days), upon addition of 4OHT myc^{Δ40-178}ER cells changed morphology within several days, similar to that seen in spontaneously differentiating cells (data not shown). This coincided with down-regulation of specific transcripts associated with pluripotency such as Nanog and Oct4 together with the upregulation of early mesoderm (brachyury) and neuroectoderm (Sox1) transcripts (FIG. 10A). These trends were confirmed by AP activity assays in which the percentage of AP positive colonies declined from 92% to 45% over a 6-day period in the presence of 4OHT (FIG. 10B). The data therefore indicates that abrogation of c-myc activity is incompatible with maintenance of ES cell self-renewal. This is consistent with the results showing that c-myc has a role in self-renewal and maintenance of pluripotency.

### Example 10

### A Role for c-myc in Maintenance of hESCs

The previous Examples demonstrate that maintained c-myc activity can sustain self-renewal of mESCs in the absence of LIF. To address this question in hESCs, clonal hES BG01 cell lines transfected with a human c-myc^{T58A}ER gene fusion were generated, identical to that used in the previous mESC studies. A 4OHT titration was performed to establish the highest concentration of drug that did not promote c-myc-dependent cell death in each of 4 cell lines. Approximately 1-5 nM 4OHT was found to be optimal for the cell line used herein. Under these conditions, negligible apoptosis and robust cell division rates comparable to the parental BG01 line were observed.

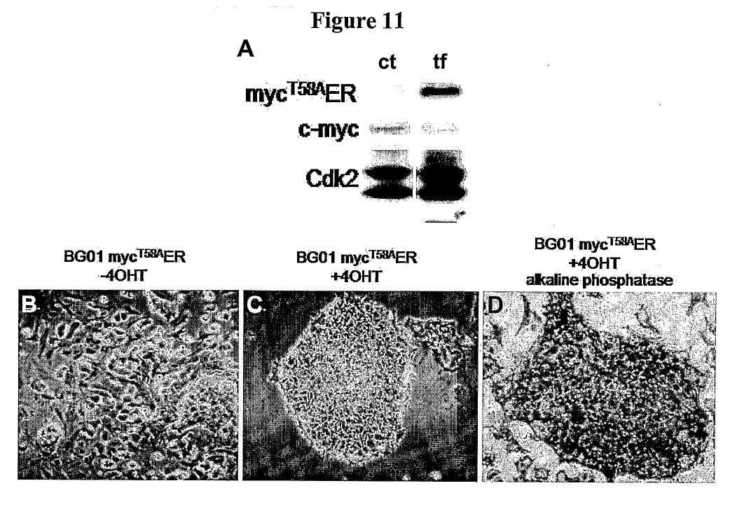

To establish a role for c-myc in HESC self-renewal, c-myc activity was enforced by the addition of 4OHT. This experiment was performed to determine whether c-myc could maintain normal HESC growth and morphology on Matrigel for a period of approximately 15 days (3 passages) in the absence of MEF-CM and bFGF, conditions where spontaneous differentiation would be anticipated. Uninduced (−40HT) cells overtly differentiated after approximately 5-6 days in a manner closely resembling untransfected hESCs when grown in the absence of MEF-CM and bFGF on Matrigel, as judged by changes in cell/colony morphology, increased cell spreading, and a reduction in cell packing density (FIGS. 11B and C and data not shown). The cell lines were also analyzed by immunoblot analysis, evaluating levels of c-myc^{T58A}ER, c-myc, and cdk2 (FIG. 11A).

Alkaline phosphatase (AP) activity was used as a read-out to assess whether c-myc maintained hESCs were still pluripotent. 67% of colonies grown with 4OHT were AP positive in contrast to −4OHT cells where only 4% were positive after a 15 day period (FIG. 11D and data not shown). As in mouse ES cells, elevated c-myc activity caused significant cell death (data not shown).

Although the cell line described expresses slightly elevated levels of the c-myc^{T58A}ER fusion, the low levels of 4OHT used in these experiments only activate a small pool of the latent cytoplasmic protein. Additional cell lines are characterized to confirm that only a small proportion of c-myc^{T58A}ER localizes to the nucleus in the presence of 1 nM 4OHT.

While the c-myc-GSK3β pathway appears to be conserved between mouse and human ESCs, it is unclear what molecule(s) transcriptionally control c-myc given that LIF-STAT3 is dispensable for hESC self-renewal. The most obvious possibility is that factors in MEF-CM or bFGF are involved. Additionally, PI3-kinase is known to activate Akt, which phosphorylates and inhibits GSK3β activation, thus preventing the destabilization of c-myc. While LIF-STAT3 signaling does not apply to self-renewal of hESCs, common elements of self-renewal including roles for c-myc and GSK3β are conserved between human and murine ESCs.

### Example 11

### A Common Mechanism of ES Cell Maintenance

The examples herein show that under conditions where ES cells are maintained, c-myc levels are elevated, T58 is unphosphorylated and GSK3β activity is suppressed. The findings herein implicate c-myc to be a central regulator of self-renewal in murine and human ES cells. Although upstream signaling can differ between murine and primate ES cells, it is proposed that pathways in the two cell types suppress GSK3β activation so as to establish conditions where c-myc has an extended half-life.

1. A method for stabilizing the pluripotency of a cell, comprising:
a) providing a human pluripotent cell; and
b) activating transcription of c-myc in the cell, to thereby stabilize the pluripotency of the cell.

2. The method of claim 1, wherein c-myc is activated independent of LIF signaling and independent of Wnt signaling.

3. The method of claim 1, wherein the human pluripotent cell is selected from the group consisting of an embryonic stem cell, an ICM/epiblast cell, a primitive ectoderm cell, a primordial germ cell, and an embryonal carcinoma cell.

4. The method of claim 1, wherein the human pluripotent cell is a human embryonic stem cell.

5. The method of claim 1, further comprising a step of culturing the cell in serum containing medium.

6. The method of claim 1, further comprising a step of using an activator of PI3-kinase signaling.

7. The cell produced by the method of claim 1.

8.-21. (canceled)

22. A method for stabilizing the pluripotency of a cell, comprising:
c) providing a pluripotent cell;
d) stabilizing c-myc protein in the cell;
to thereby maintain the pluripotency of the cell.

23. The method of claim 22, wherein the pluripotent cell is a mouse cell.

24. The method of claim 22, wherein the pluripotent cell is a human cell.

25. The method of claim 24, wherein the human cell is selected from the group consisting of an embryonic stem cell, an ICM/epiblast cell, a primitive ectoderm cell, a primordial germ cell, and an embryonal carcinoma cell.

26. The method of claim 25, wherein the human cell is a human embryonic stem cell.

27. The method of claim 22, further comprising a step of culturing the cell in serum containing medium.

28. The method of claim 22, wherein c-myc is stabilized by contacting the cell with an activator of PI3-kinase signaling.

29. The method of claim 22, wherein c-myc is stabilized by inhibiting the activity of GSK3β.

30. The method of claim 29, wherein GSK3β signaling is inhibited by contacting the cell with BIO.

31. The method of claim 29, wherein GSK3β signaling is inhibited by inhibiting c-myc T58 phosphorylation.

32. The method of claim 29, wherein GSK3β signaling is inhibited through inhibiting the Pin1/PP2A signaling mechanism.

33. The method of claim 22, wherein c-myc is stabilized through an interaction with Max.

34. The cell produced by the method of Claim 22.

35.-63. (canceled)
